(19) United States
(12) Patent Application Publication
(10) Pub. No.: US 2015/0376710 A1
(43) Pub. Date: Dec. 31, 2015

(54) METHODS OF EVALUATING RESPONSE TO CANCER THERAPY

(71) Applicant: THE BOARD OF REGENTS OF THE UNIVERSITY OF TEXAS SYSTEM, Austin, TX (US)

(72) Inventors: Christos HATZIS, Melrose, MA (US); W. Fraser SYMMANS, Houston, TX (US)

(21) Appl. No.: 14/633,987

(22) Filed: Feb. 27, 2015

Related U.S. Application Data
(63) Continuation of application No. 13/641,057, filed on Dec. 14, 2012, now abandoned.
(60) Provisional application No. 61/324,166, filed on Apr. 14, 2010.

(51) Int. Cl. C12Q 1/68; G06F 19/00; G01N 33/574

(52) U.S. Cl. CPC C12Q 1/6886; G01N 33/57492; G06F 19/34; C12Q 2600/158; C12Q 2600/112; C12Q 2600/106; G01N 2333/91205

(57) ABSTRACT

A method of evaluating a cancer patient comprising evaluating gene expression levels in a patient sample, calculating a predictor score using the gene expression levels, and assessing the likelihood of a therapeutic outcome using the predictor score is disclosed.

This application claims priority to U.S. Provisional application Ser. No. 61/324,166 filed Apr. 14, 2010, which is incorporated herein by reference in its entirety.

### BACKGROUND OF THE INVENTION

### I. Field of the Invention

Embodiments of this invention are directed generally to biology and medicine. In certain aspects the invention relates to a gene set whose levels of expression are evaluated and used to prognose and/or derive a survival indicator for a patient who has undergone therapy, who is undergoing therapy, or who is a candidate for therapy.

### II. Background

There are four main approaches to improving the ability to predict responsiveness to therapies. One approach is a standard predictive or chemopredictive study focused on treatment, in which a sufficiently powered discovery population of subjects is used to define a predictive test that must then be proven to be accurate in a similarly sized validation population (Ransohoff, 2005; Ransohoff 2004). Several studies have used this approach to define predictive genes for adjuvant tamoxifen therapy (Ma et al., 2004; Jansen et al., 2005; Loi et al., 2005). There are advantages to this approach, particularly when samples are available from mature studies for retrospective analysis. But two disadvantages are that the study design is empirical and that adjuvant (post surgery) treatment introduces surgery as a confounding variable, because it is impossible to ever know which patients were cured by their surgery and would never relapse, irrespective of their sensitivity to systemic therapy. Neoadjuvant chemotherapy trials enable a direct comparison of tumor characteristics with pathologic response to the specific therapy (Ayers et al., 2004).

### SUMMARY OF THE INVENTION

In medicine today, doctors search for methods of predicting how a patient (given their condition) may respond to treatment. Symptoms and tests may indicate favorable treatment with standard therapies. Likewise, a number of symptoms, health factors, and tests may indicate a less favorable treatment result with standard treatment—this may indicate that a more aggressive treatment plan may be desired. Prognostic scoring is also used for cancer outcome predictions.

Although pathologic complete response (pCR) has been adopted as the primary endpoint for neoadjuvant trials because it is associated with long-term survival, it has not been uniformly or consistently defined (Bear, 2006; Carey, 2005; Hennessy, 2005; Kaufmann, 2006; Kuroi, 2005; Kurosumi, 2004; Rajan, 2004; von Minckwitz, 2005). While it is generally agreed that a definition of pCR should include patients without residual invasive carcinoma in the breast (pT0), the presence of nodal metastasis, minimal residual cellularity, and residual in situ carcinoma are not consistently stated as either pCR or residual disease (RD) (Bear, 2006; Kaufmann, 2006; Hennessy, 2005; Rajan, 2004). Therefore, dichotomization of response as pCR or residual disease (RD) may be simplistic for the objective of assay discovery and validation, particularly because residual disease (RD) after neoadjuvant treatment includes a broad range of actual tumor shrinkage. In some patients who are categorized as RD but actually show minimal residual disease, the response outcome blurs the prognostic distinction between pCR and RD. On the other hand, it should be possible to clearly identify patients within RD who are resistant to treatment in order to develop management strategies for this adverse outcome.

Expression markers are chosen for the ability to classify and/or identify patients as to probability for response (or non response) to therapy. Response to therapy is commonly classified by the RECIST criteria established by the World Health Organization, the National Cancer Institute and the European Organization for Research and Treatment of Cancer. The RECIST criteria classify response as progressive disease (PD), stable disease (SD), partial response (PR), and complete response (CR). A good response is typically considered to include PR+CR (collectively referred to herein as Objective Response).

Certain aspects of the invention include methods of evaluating a cancer patient comprising one or more of the steps of (a) evaluating gene expression levels in a patient sample comprising cancer cells or an RNA sample isolated from one or more a patient samples, wherein a plurality of genes to be evaluated are selected from 2, 3, 4, 5, 6, 7, 8, 9, 10, 11, 12, 13, 14, 15, 20, 25, 30, 40, 50, 60, 70, 80, 90, 100, 150, or all of the genes identified in Table 2, Table 3, and Table 4, including all ranges and values there between and all subsets and combinations thereof (5, 10, 15, 20, 25, 100 or more such genes can be specifically excluded, including all values and ranges there between); (b) calculating a predictor score using a gene expression profile index; and (c) assessing the likelihood of a therapeutic outcome using the predictor score. The method may further comprise classifying a patient prior to evaluation. In certain aspects classification can include identifying a cancer patient with a disease state classified as a residual disease state or other clinically defined state prior to evaluation. In certain aspects, a predictor includes but is not limited to a measure for distant relapse-free survival (DRFS).

In still a further aspect, a gene expression index comprises 2, 3, 4, 5, 6, 7, 8, 9, 10, 11, 12, 13, 14, 15, 20, 25, 30, 40, 50, 60, 70, 80, 90, 100, 150 or all of the genes identified in Table 2, Table 3, and Table 4 including all values and ranges there between as well as a number of subsets of these genes which may include some genes from one or more tables and exclude others from the same table or other tables.

In other aspects, a patient may be stratified or analyzed by using other factors such as protein expression, demographic information, family history, and other biological or medical states. The method may include determining Her2-neu and/or estrogen receptor status of the patient sample and/or evaluation of tumor size, cellularity of tumor bed, and/or nodal burden to name a few.

The methods may also provide a treatment recommendation depending on the assessment derived from analysis of the gene expression profile as well as other factors. In certain aspects the recommendation may be based on residual cancer burden (RCB) classification or the like. A treatment is typically a standard treatment or a more aggressive non-standard treatment depending on the analysis. For example a treatment may be combination of one or more cancer therapies, such as hormonal therapy and/or chemotherapy. Hormonal therapy includes, but is not limited to tamoxifen therapy, aromatase inhibitor therapy, or SERM therapy.

In other aspects, preparing a gene expression index can include one or more of the following steps: (a) obtaining data associated with a plurality of cancer patients, such as breast cancer, melanoma, ovarian cancer, testicular cancer or the like comprising measuring expression levels of a plurality of genes in samples from a plurality of patients; (b) partitioning the data into a first and second dataset; (c) evaluating the data and identifying data associated with a particular treatment outcome; (d) selecting a set of genes whose expression levels are indicative of therapeutic outcome. In one aspect, the index includes evaluation of survival of the patient population sampled for all or part of the reference population of tumor samples such as the distant relapse-free survival (DRFS) of the patient population.

Other aspects of the invention include kits to determine responsiveness of a cancer or cancer patient to a treatment or therapy comprising one or more of (a) reagents for determining expression levels of a plurality of genes selected from Table 2, Table 3, and Table 4 or combinations thereof, such as probe sets that identify and measure the levels of gene transcripts, transcription, or protein levels; and software encoding methods for designing, gathering, inputting, analyzing and/or assessing various data, which includes an algorithm for calculating a predictor score based on the analysis of the gene expression levels.

In still other aspects the invention includes an apparatus, or system for providing assessment of a sample relative to a gene expression index, the system comprising (a) an application server comprising an input manager to receive expression data from a user for a plurality of genes selected from Table 2, Table 3, and Table 4 or combinations thereof obtained from a patient sample or an RNA sample from such patient sample; and (b) a network server comprising an output manager constructed and arranged to provide an assessment to the user.

In yet another aspect the invention includes a computer readable medium having software modules for performing the one or more of the methods described herein comprising the acts of: (a) comparing gene expression data obtained from a patient sample for a plurality of genes selected from Table 2, Table 3, and Table 4 or combinations thereof with a reference; and (b) providing a predictor score to a physician for use in determining an appropriate therapeutic regimen for a patient.

In still yet another aspect the invention includes a computer system, having a processor, memory, external data storage, input/output mechanisms, a display, for performing the method of the invention, comprising (a) a database; (b) logic mechanisms in the computer for generating the transcriptional profile index; and (c) a comparing mechanism in the computer for comparing the gene expression reference to expression data from a patient sample or an RNA sample from such a patient sample to calculate a predictor score.

An internet accessible portal may be use to provide biological information constructed and arranged to execute a computer-implemented methods for providing: (a) a comparison of gene expression data of a plurality of genes of claim 1 in a patient sample with a transcriptional profile index; and (b) providing a predictor score to a physician for use in determining an appropriate therapeutic regime for a patient.

Other embodiments of the invention are discussed throughout this application. Any embodiment discussed with respect to one aspect of the invention applies to other aspects of the invention as well and vice versa. The embodiments in the Example section are understood to be embodiments of the invention that are applicable to all aspects of the invention.

The terms “inhibiting,” “reducing,” or “prevention,” or any variation of these terms, when used in the claims and/or the specification includes any measurable decrease or complete inhibition to achieve a desired result.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 Plot of relapse-free survival in predicted responders and non-responders using the relapse-based predictor of Example 2 in the validation cohort of patients.

FIG. 2 Plot of distant relapse-free survival outcomes in predicted responders and non-responders using response-based endpoint of RCB0/I of Example 4 in the validation cohort of patients.

FIG. 3A-3B Prediction of responders to chemotherapy in ER-positive tumors (A) and ER-negative tumors (B) using the response-based predictor in the validation cohort of patients.

FIG. 4 Prediction of responders to chemotherapy using a combination of relapse- and response-based predictors in the validation cohort of patients.

FIG. 5A-5B Prediction of responders to chemotherapy in ER-positive tumors (A) and ER-negative tumors (B) using the combination of relapse- and response-based predictors in the validation cohort of patients.

FIG. 6 Endocrine sensitivity index in the validation cohort of patients.

FIG. 7 Plot of combined predictions in the validation cohort to identify responders and non-responders to chemotherapy.

FIG. 8A-8B Plot of distant relapse-free survival within ER-specific subsets of the validation cohort, (A) ER-positive patients stratified by predicted responders and non-responders, (B) ER-negative patients stratified by predicted responders and non-responders.

FIG. 9 The decision algorithm that was used in the genomic test to predict a patient's sensitivity to adjuvant chemotherapy or chemo-endocrine therapy from a biopsy of newly diagnosed invasive breast cancer. (*) predicted sensitivity to endocrine therapy was defined as high or intermediate genomic sensitivity to endocrine therapy (SET) index; (**) predicted resistance to chemotherapy was defined as predicted extensive residual cancer burden (RCB-III) or predicted distant relapse or death within 3 years of diagnosis; (***) predicted sensitivity to chemotherapy was defined as predicted pathologic complete response (pCR) or minimal residual cancer burden (RCB-I).

FIG. 10A-10C Plot of responders and non-responders in the validation cohort of patients predicted by using a combination of predictors of relapse, response as RCB-0/I, resistance as RCB-III, and SET. Kaplan-Meier estimates of distant relapse-free survival according to genomic predictions (before treatment) as treatment-sensitive (Rx Sensitive) or treatment-insensitive (Rx Insensitive) in the discovery (A) and independent validation (B) cohorts. For comparison, the prognosis of the groups stratified by actual pathologic response (pathologic complete response vs. residual disease) after completion of all chemotherapy is shown for the validation cohort (C). P-values are from the log-rank test. Vertical ticks on the curves indicate censored observations.

FIG. 11A-11D Subset analysis of genomic predictions in the validation cohort: ER+/HER2− (A), ER−/HER2− (B), taxane chemotherapy administered as 12 cycles of weekly paclitaxel (C) or 4 cycles of 3-weekly docetaxel (D). P-values are from the log-rank test. Vertical ticks on the curves indicate censored observations.

FIG. 12A-12H Kaplan-Meier estimates of distant relapse-free survival in the discovery cohort (A-D) and the independent validation cohort (E-H) of patients treated with sequential taxane-anthracycline chemotherapy, then endocrine therapy if hormone receptor-positive, stratified by other signatures reported to be predictive of response to neoadjuvant taxane-anthracycline chemotherapy. A prognostic signature for genomic grade index predicts pathologic response if high GGI versus low GGI (A, E); the intrinsic subtype classifier predicts pathologic response if basal-like or luminal B versus other subtypes (B, F); a genomic predictor of pathologic complete response (pCR) versus residual disease following taxane-anthracycline chemotherapy (C, G); and the genomic predictor of excellent pathologic response (pCR or RCB-I) versus other residual disease, according to ER status, that we incorporated in the last step of our prediction algorithm (D, H). P-values are from the log-rank test. Vertical ticks on the curves indicate censored observations.

FIG. 13 Schematic of use of the predictor assay to guide decisions in therapy outcome.

### DETAILED DESCRIPTION OF THE INVENTION

Despite the critical importance of selecting the most effective adjuvant/neoadjuvant chemotherapy for an individual, diagnostic tests to guide selection of the optimal regimen for a particular patient continue to be inadequate (Carlson, 2000; Goldhirsch, 2003). Estrogen receptor (ER) negative status, high grade and high proliferative activity are histological characteristics that tend to indicate more chemotherapy sensitive cancer (Bast, 2001; Ross, 2003; Rouzier, 2005). However, although these clinicopathologic variables may identify eligibility or predict general chemotherapy sensitivity, they have little potential to guide selection of a specific treatment regimen in standard-of-care practice.

The limited utility of individual markers to predict clinical outcome of cancer may be due to the incomplete understanding of the function of these markers. In addition, biologically important molecules act in concert and form complex, interactive pathways where an individual molecule may only contribute limited information on the functional activity of a whole pathway. The promise of microarray technology is that, by assessing the transcriptional activity of a large number of genes, the complex gene-expression profile may contain more information than any individual marker that contributes to it.

There are examples indicating that the molecular classification of cancer based on gene-expression profiles could be important in framing patient management strategies. Unsupervised clustering of breast cancer specimens consistently separated tumors into ER⁺ and ER⁻ clusters (Gruvberger, 2001; Perou, 2000; Pusztai, 2003). Analysis of gene-expression profiles also distinguished sporadic breast cancers from breast cancer gene, BRCA, mutant cases (Hedenfalk, 2001). Transcriptional profiles have also revealed previously unrecognized molecular subgroups within existing histological categories in breast cancer (Perou, 2000), diffuse large-B-cell lymphoma, and soft tissue and central nervous system embryonal tumors (Nielsen, 2002; Pomeroy, 2002). In addition, gene-expression profiles have been shown to predict survival of patients with node-negative breast cancer (van de Vijver, 2002; van 't Veer, 2002), lymphoma (Alizadeh, 2000; Rosenwald, 2002), renal cancer (Takahashi, 2001), and lung cancer (Beer, 2002).

Previous efforts into applying gene expression-based predictors in breast cancer have focused largely on predicting a patient's risk of cancer recurrence in the event of either receiving no systemic treatment after surgery (van de Vijver, 2002; van 't Veer, 2002; Wang, 2005) or receiving tamoxifen, a hormonal therapy agent, for 5 years after surgery (Paik, 2006; Paik, 2004; Ma, 2006; Davis, 2007). These gene-based predictors do not directly address the need or the responsiveness to chemotherapy although a high risk of recurrence may indirectly suggest the general consideration of chemotherapy among the available options for patient management.

Other research efforts have also reported gene-based predictors of response to standard breast cancer treatments (Ayers, 2004; Bild, 2006; Chang, 2003; Hess, 2006; Modlich, 2006) although these are not commercially marketed yet as assays. Some of these predictors are developed using patient tissue samples treated clinically with a specific chemotherapy regimen and subsequently comparing genomic profiles of responders versus non-responders using survival-driven endpoints (Ayers, 2004; Chang, 2003; Hess, 2006; Modlich, 2006) whereas others are focused on analyses of changes in genes within breast cancer cell lines that are treated in vitro with single standard therapeutic agents (Bild, 2006).

As an in vivo model for marker development and validation, neoadjuvant (preoperative) chemotherapy provides an opportunity to gain access to samples that directly describe tumor response to therapy. Furthermore, complete eradication of all invasive cancer from the breast and regional lymph nodes, called pathologic complete response (pCR), is associated with excellent long-term cancer-free survival (Fisher, 1998; Kuerer, 1999). Therefore, the goal in developing treatment-directed response markers is to evaluate gene expression profiles in order to predict who may achieve pCR versus residual disease (RD). Pathologic CR is a meaningful clinical end-point to predict because these patients experience prolonged disease-free and overall survival compared to patients with lesser response (Cleator, 2005; Fisher, 1998; Kaufmann, 2006; Wolmark, 2001). Good survival in these patients reflects benefit from chemotherapy since most clinical and gene expression variables that are associated with pCR (i.e., high grade, ER-negative status, high OncotypeDX recurrence score) tend to predict worse prognosis in the absence of chemotherapy (Paik, 2006; Paik, 2004).

Previous work has demonstrated the development and validation of a 30-probe genomic predictor for response to a taxane-containing chemotherapy (Ayers, 2004; Hess, 2006). The treatment administered in the neoadjuvant setting was sequential paclitaxel anthracycline preoperative chemotherapy (T/FAC). A complex multidrug regimen was selected for study because combination chemotherapy represents the current clinical standard for patients who require systemic cytotoxic treatment. Also, studies that explore gene signatures for response to individual drugs may not fully capture sensitivity to combination chemotherapy as practiced in standard-of-care.

A cohort of 82 patients was used for predictor discovery of pCR to preoperative T/FAC chemotherapy using fine needle biopsies taken before treatment and by analyzing gene profiles generated from a commercially available standard gene expression profiling technology (Affymetrix, Santa Clara, Calif.). Although several analytic techniques and resulting gene sets for response prediction were studied, the nominally best predictor for pCR with the least number of genes, called DLDA-30, was selected for independent validation in 51 additional patients. The predictor showed substantially higher sensitivity (a measure of how well a predictor identifies responsiveness or non-responsiveness to a therapy, e.g., true positives/(true positives+false negatives)) (92% vs. 61%) and slightly better negative predictive value (NPV, the proportion of patients with negative test results who are correctly diagnosed.) (96% vs. 86%) than a clinical predictor based on ER, grade and age (Hess, 2006). The positive predictive value (PPV, is the proportion of patients with positive test results who are correctly diagnosed.) of the genomic predictor at 52% (95 CI: 30%-73%), was significantly higher than the baseline 26% pCR rate in unselected patients. A sensitivity of 100% means that the test recognizes all patient as either responsive to therapy or non-responsive to therapy. Typically, sensitivity alone does not tell us how well the test predicts other classes (that is, about the negative cases). Sensitivity is not the same as the positive predictive value (ratio of true positives to combined true and false positives), which is as much a statement about the proportion of actual positives in the population being tested as it is about the test. The calculation of sensitivity typically does not take into account indeterminate test results. If a test cannot be repeated, the options are to exclude indeterminate samples from analyses (but the number of exclusions should be stated when quoting sensitivity), or, alternatively, indeterminate samples can be treated as false negatives (which gives the worst-case value for sensitivity and may therefore underestimate it).

Although this predictor and others described in literature (Chang, 2003; Modlich, 2006) may help define a patient population that is more likely to achieve pCR than the general patient population, further developments can help refine prediction of treatment response considerably. Although pCR as a response endpoint is strongly correlated with high treatment-related survival, patients with residual disease (RD) after treatment encompass a wide range of outcomes ranging from very good prognosis (“near-pCR”) to drug resistance. Predictors that can better classify response outcomes to capture and differentiate the high responders and non-responders within the spectrum of residual disease could significantly benefit patient management.

Although pathologic complete response (pCR) has been adopted as the primary endpoint for neoadjuvant trials because it is associated with long-term survival, it has not been uniformly or consistently defined (Bear, 2006; Carey, 2005; Hennessy, 2005; Kaufmann, 2006; Kuroi, 2005; Kurosumi, 2004; Rajan, 2004; von Minckwitz, 2005). While it is generally agreed that a definition of pCR should include patients without residual invasive carcinoma in the breast (pT0), the presence of nodal metastasis, minimal residual cellularity, and residual in situ carcinoma are not consistently stated as either pCR or residual disease (RD) (Bear, 2006; Kaufmann, 2006; Hennessy, 2005; Rajan, 2004). Therefore, dichotomization of response as pCR or residual disease (RD) may be simplistic for the objective of assay discovery and validation, particularly because residual disease (RD) after neoadjuvant treatment includes a broad range of actual tumor shrinkage. In some patients who are categorized as RD but actually show minimal residual disease, the response outcome blurs the prognostic distinction between pCR and RD. On the other hand, it should be possible to clearly identify patients within RD who are resistant to treatment in order to develop management strategies for this adverse outcome.

A measure of residual disease or residual cancer burden (RCB), previously developed and reported, may be useful as a variable to characterize response to treatment (Symmans et al., 2007). This measure is derived from the primary tumor dimensions, cellularity of the tumor bed, and axillary nodal burden. Each component contributes meaningful pathologic information and can be obtained using routine pathologic materials and methods of interpretation that could easily be implemented in routine diagnostic practice. RCB measurements can provide a continuous parameter of residual disease and thus of response or resistance, so that all subject responses contribute to the analysis.

RCB is divided into four survival-related classes (RCB-0 to RCB-III) where patients with minimal residual disease (RCB-I) have the same 5-year relapse-free survival as those with pCR (RCB-0), irrespective of the type of neoadjuvant chemotherapy administered, adjuvant hormonal therapy or the pathologic stage of RD. Therefore, the combination of RCB-0 (pCR) and RCB-I expands the subset of patients who can be identified as having “good response” and to have benefited from the chemotherapy. Extensive residual disease (RCB-III), on the other hand, is associated with poor prognosis, irrespective of the type of neoadjuvant chemotherapy administered, adjuvant hormonal therapy, or the pathologic stage of RD. In particular, all patients with RCB-III after T/FAC chemotherapy, who did not receive adjuvant hormonal therapy, suffered distant relapse within 3 years (Symmans et al., 2007). This identifies an important subset of patients who are not responsive to chemotherapy, or with residual disease (after surgery) that is too extensive to be controlled by hormonal therapy alone.

Therefore, residual cancer burden (RCB) is an informative tool and a metric to help develop response predictors based on better characterization of likely treatment outcomes.

RCB categories can be employed with existing methods to define surrogate endpoints from neoadjuvant trials. As a metric correlated with survival, RCB is strongly and independently prognostic and the classes of RCB capture distinct sets of survival-based outcomes. Development of a predictor that reports likelihood of a patient's tumor post-treatment to belong to one of the RCB classes, rather than simply pCR as an endpoint, can yield valuable diagnostic information for efficient treatment management. In certain aspects, predictors specific to RCB-0 (pCR or complete response), RCB-0/I (pCR+near-pCR called good response) and RCB-III (resistance) are developed. In certain aspects of the methods described, the inventors have also accounted for tumor sub-types based on the status of two receptors, Her2-neu and ER, allowing for the predictors to capture heterogeneity within breast cancers and achieve acceptable diagnostic performance.

### III. Predictors of Response or Resistance to Therapy

Sets of genes are defined that are prognostic, diagnostic, or predictive or indicative of the outcome for a cancer patient. These genes can be incorporated into an index or predictor of such an outcome and used in the management of the treatment for a given patient. Prognosis is a medical term denoting the doctor's prediction of how a patient's disease will progress, and whether there is chance of recovery.

Outcome can be represented in various forms to indicate probability of survival or likely survival outcome. In biostatistics, survival rate is a part of survival analysis, indicating the percentage of people in a study or treatment group who are alive for a given period of time after diagnosis. Survival rates are important for prognosis; for example, whether a type of cancer has a good or bad prognosis can be determined from its survival rate or survival outcome.

Patients with a certain disease can die directly from that disease or from an unrelated cause such as a car accident. When the precise cause of death is not specified, this is called the overall survival rate or observed survival rate. Doctors often use mean overall survival rates to estimate the patient's prognosis. This is often expressed over standard time periods, like one, five, and ten years. For example, prostate cancer has a much higher one year overall survival rate than pancreatic cancer, and thus has a better prognosis.

When someone is more interested in how survival is affected by the disease, there is also the net survival rate, which filters out the effect of mortality from other causes than the disease. Typically, the two main ways to calculate net survival are relative survival and cause specific survival or disease specific survival.

Relative survival is calculated by dividing the overall survival after diagnosis of a disease by the survival as observed in a similar population that was not diagnosed with that disease. A similar population is composed of individuals with at least age and gender similar to those diagnosed with the disease. Cause-specific survival is calculated by treating deaths from other causes than the disease as withdrawals from the population that don't lower survival, comparable to patients who are not observed any longer, e.g. due to reaching the end of the study period. Relative survival has the advantage that it does not depend on accuracy of the reported cause of death; cause-specific survival has the advantage that it does not depend on the ability to find a similar population of people without the disease.

Survival is not the only endpoint that can be used as a metric in developing predictors such as those described herein. Endpoints or therapeutic outcomes can include survival or distant relapse-free survival (DRFS). Other endpoints are discussed in Cooper and Kaanders, Biological surrogate end-points in cancer trials: Potential uses, benefits and pitfalls, European Journal of Cancer, Volume 41, Issue 9, Pages 1261-1266, which is incorporated herein by reference. A “surrogate marker” or “surrogate endpoint” or “secondary endpoint” typically will refer to a biological or clinical parameter that is measured in place of the biologically definitive or clinically most meaningful parameter, i.e., survival. Primary endpoints may also include limitation of pharmacologic therapies, reduction of time to death, or reduction in the progression of the disease, disorder, or condition. Surrogate markers are pathophysiologic parameters determined by medical or clinical laboratory diagnosis that are associated and have been correlated with the prognosis, progression, predisposition, or risk analysis with a disease, disorder, or condition that are not directly related to the primary diagnosed pathophysiologic condition. Secondary endpoints are those that supplement the primary endpoint. For example, secondary endpoints include reduction in pharmacologic therapy, reduction in requirement of a medical device, or alteration of the progression of the disease disorder, or condition. Typically, a clinical endpoint may refer to a disease, symptom, or sign that constitutes one of the target outcomes of the therapy or clinical trial. The results of a therapy or clinical trial generally indicate the number of people enrolled who reached the pre-determined clinical endpoint during the study interval, compared with the overall number of people who were enrolled. Once a patient reaches the endpoint, he or she is generally excluded from further experimental intervention (the origin of the term endpoint). For example, a clinical trial investigating the ability of a medication to prevent heart attack might use chest pain as a clinical endpoint. Any patient enrolled in the trial who develops chest pain over the course of the trial, then, would be counted as having reached that clinical endpoint. The results would ultimately reflect the fraction of patients who reached the endpoint of having developed chest pain, compared with the overall number of people enrolled. When an experiment involves a control group, the fraction of individuals who reach the clinical endpoint after an intervention is compared with the fraction of individuals in the control group who reached the same clinical endpoint, thus reflecting the ability of the intervention to prevent the endpoint in question. Some studies will examine the incidence of a combined endpoint, which can merge a variety of outcomes into one group.

When building prediction rules of treatment response or disease state in general from gene expression data can be selected from a small subset of informative genes that will be used as prognostic features in the predictor. Most predictors employ univariate filtering to rank the candidate genes according to the p-value of a two-sample unequal variance t-test comparing the mean expression values of each gene in the two response classes (e.g., pCR and RD). Univariate filtering methods have the disadvantage that they do not deal well with redundant features (genes that have similar expression profiles) and therefore the resulting predictors tend to be less robust (Lai, 2006).

The method used to identify predictive genes involved first, applying a filter to the gene expression data of all probes on an array to select the top probe sets to be used in signature development using the above described algorithm. Gene filtering can be based on the regularized t-test for the selected response endpoint such as pCR or RCB-0 (complete response), RCB-0/I (good response), or RCB-III (poor response). Other methods for gene filtering include methods that utilize non-specific global filtering criteria. These include, but are not limited to intensity-based filtering, which aims to remove genes that are not expressed at all in the samples studied or variability-based filtering, which aims to remove genes with low variability across samples.

A multivariate method was used to simultaneously select the signature genes and to calculate the classification score. The predictor is determined by level of penalization, which determines the number of genes included in the predictive signature, and the choice of a decision threshold to dichotomize the classification score. As one example, the inventors selected the maximum level of penalization resulting in the smallest signatures that yield significant cross-validated predictor or outcome predictor, each of these terms can be used interchangeably, performance—this step determines the signature probe sets and their weights. Then, a decision threshold is selected in order to optimize the predictive values of the predictor. Evaluation of the predictors was based on the joint confidence interval of the positive predictive value (PPV) and the negative predictive value (NPV) of the predictor at 5% significance level (low 95% confidence limit of PPV≧baseline response rate & low 95% confidence limit of NPV≧1—baseline response rate).

In developing the RCB-based predictor, the inventors used an approach that combines feature selection and model discovery using a multivariate penalized approach, an example of which is Gradient Directed Regularization developed by Prof J. Friedman at Stanford University, a description of which can be found on the World Wide Web at stat.stanford.edu/˜jhf/ftp/pathlite.pdf. Typically, the informative genes are selected with penalization using the maximization of the area under the receiver operating characteristic (ROC) curve (AUC) as the optimization criterion. Ma and Huang have previously used a similar approach for disease classification (Ma, 2006). A receiver operating characteristic (ROC), or simply ROC curve, is a graphical plot of the sensitivity vs. (1—specificity) for a binary classifier system as its discrimination threshold is varied. The ROC can also be represented equivalently by plotting the fraction of true positives (TPR=true positive rate) vs. the fraction of false positives (FPR=false positive rate). The best possible prediction method would yield a point in the upper left corner or coordinate (0,1) of the ROC space, representing 100% sensitivity (all true positives are found) and 100% specificity (no false positives are found). The (0,1) point is also called a perfect classification. A completely random guess would give a point along a diagonal line (the so-called line of no-discrimination) from the left bottom to the top right corners. The diagonal line divides the ROC space in areas of good or bad classification/diagnostic. Points above the diagonal line indicate good classification results, while points below the line indicate wrong results.

As an example of predictor discovery and evaluation the protocol suggested by Wessels et al. was followed (Wessels, 2005). The methodology is briefly explained below. First, the input dataset is randomly partitioned into a training set and a test set. A 3-fold cross-validation based on Dudoit et al. recommendation of a 2:1 split between training and test sets was used (Dudoit, 2002). The training set consisting of ⅔ of the original data is used to develop a predictor. To account for bias in the several data-dependent decisions involved in building the predictor, a 5-fold internal cross-validation can be used to select the optimal set of genes for the predictor and to tune the parameters of the predictor, e.g., the degree of penalization. Since different optimal reporter gene sets might result from the different internal cross-validation folds, the number of times each gene is selected is tracked to provide a measure of its importance or its reliability. The trained predictor is then tested on the ⅕ hold-out part of the training dataset and its performance is evaluated based on the AUC.

To obtain a less biased estimate of classification performance, the trained predictor or outcome predictor can be evaluated on the test set (⅓ of the original data) that was not used in training the predictor. To assess the significance of the predictive performance of the trained predictor, the permutation predictive performance of the predictor was estimated by randomly scrambling the outcome labels in the test dataset. The entire process of randomly splitting the data to a training and a test set was repeated a number of times to obtain the distributions and summary statistics of the performance metrics.

Typically, under cross-validation the decision threshold is varied along all possible values and for each value predictor performance (accuracy, positive predictive value (PPV), negative predictive value (NPV)) is determined. The threshold is selected that yields the best compromise between PPV and NPV, as typically increasing PPV results in decreasing NPV. Typically, the objective is to maximize both.

In certain aspects, other measurements or determinations can be made in conjunction the nucleic acid analysis, for example determination of protein expression and/or histology of a sample. Protein expression can be detected in tumor tissue, cell material obtained by biopsy and the like. For example, a biopsy sample can be immobilized and contacted with an antibody, an antibody fragment or an aptamer that binds selectively to the protein to be detected. The sample can be assayed to determine whether the antibody, fragment or aptamer has bound to the protein by techniques well known in the art. Protein expression can be measured by a variety of methods including but not limited to Western blot, immunoblot, enzyme-linked immunosorbant assay (ELISA), radioimmunoassay (RIA), immunoprecipitation, surface plasmon resonance, immunohistochemical (IHC) analysis, mass spectrometry, fluorescence activated cell sorting (FACS) and flow cytometry.

In a further aspect, IHC analysis is used to measure protein expression. The level of expression for a sample is determined by IHC by staining the sample for a particular expression marker and developing a score for the staining. For example, monoclonal antibodies can be used to stain for the expression of a marker of interest. Mouse antibodies are known for use in the staining of the marker PTEN. Samples can be evaluated for the frequency of cells stained for each sample and the intensity of the stain. Typically, a score based on the frequency (rated from 0-4) and intensity (rated from 0-4) of the stained sample is developed as a measure of overall expression. Exemplary but non-limiting methods for IHC and criteria for scoring expression are described in detail in Handbook of Immunohistochemistry and In Situ Hybridization in Human Carcinomas, M. Hayat Ed., 2004, Academic Press.

### IV. Use of Predictor for Patient Evaluation

In one aspect of the invention, a predictor or transcriptional profile index is used to measure the expression of many genes that provide predictive information about a likely outcome for a particular patient. The invention includes the methods for standardizing the expression values of future samples to a normalization standard that will allow direct comparison of the results to past samples, such as from a clinical trial. The invention also includes the biostatistical methods to calculate and report such results. A sample as used herein can comprise any number of cells that is sufficient for a clinical diagnosis or prognosis, and typically contain at least, at most or about 100 target cells.

The microarrays provide a suitable method to measure gene expression from clinical samples. mRNA levels measured by microarrays, such as Affymetrix U133A gene chips, in fine needle aspirates (FNA), core needle biopsy, and/or frozen tumor tissue samples of breast cancer correlated closely with protein expression by enzyme immunoassay and by routine immunohistochemistry.

Estrogen Receptor and Her2-Neu Status.

ER-positive breast cancer includes a continuum of ER expression that might reflect a continuum of biologic behavior and endocrine sensitivity. Others have reported that some breast cancers are difficult to predict as ER-positive based on transcriptional profile and described non-estrogenic growth effects, such as HER-2, more frequently in this small subset of tumors with aggressive natural history (Kun et al., 2003). Indeed, ER mRNA levels are lower in breast cancers that are positive for both ER and HER2 (Konecny et al., 2003).

### V. Cancer Therapies

Diagnostic tools are needed not merely for prognosis, but, for providing a biological rationale and to demonstrate clinical benefit when they are used to guide the selection and duration of therapies, particularly in light of the cost, complexity, toxicity, benefits and other factors related to such therapies. An index or predictor can be used to predict the likelihood of response rather than intrinsic prognosis.

In addition to other know methods of cancer therapy, hormone therapies may be employed in the treatment of patients identified as having hormone sensitive cancers. Hormones, or other compounds that stimulate or inhibit these pathways, can bind to hormone receptors, blocking a cancer's ability to get the hormones it needs for growth. By altering the hormone supply, hormone therapy can inhibit growth of a tumor or shrink the tumor. Typically, these cancer treatments only work for hormone-sensitive cancers. If a cancer is hormone sensitive, a patient might benefit from hormone therapy as part of cancer treatment. Sensitive to hormones is usually determined by taking a sample of a tumor (biopsy) and conducting analysis in a laboratory.

A. Chemotherapy

Chemotherapy is the use of chemical substances to treat disease. In its modern-day use, it refers to cytotoxic drugs used to treat cancer or the combination of these drugs into a standardized treatment regimen. There are a number of strategies in the administration of chemotherapeutic drugs used today. Chemotherapy may be given with a curative intent or it may aim to prolong life or to palliate symptoms.

Combined modality chemotherapy is the use of drugs with other cancer treatments, such as radiation therapy or surgery. Combination chemotherapy is a similar practice which involves treating a patient with a number of different drugs simultaneously, e.g., T/FAC therapy. Typically, the drugs differ in their mechanism and side effects. The biggest advantage is minimizing the chances of resistance developing to any one agent.

In neoadjuvant chemotherapy (preoperative treatment) initial chemotherapy is aimed for shrinking the primary tumor, thereby rendering local therapy (surgery or radiotherapy) less destructive or more effective.

Adjuvant chemotherapy (postoperative treatment) can be used when there is little evidence of cancer present, but there is risk of recurrence. This can help reduce chances of resistance developing if the tumor does develop. It is also useful in killing any cancerous cells which have spread to other parts of the body. This is often effective as the newly growing tumors are fast-dividing, and therefore very susceptible.

Palliative chemotherapy is given without curative intent, but simply to decrease tumor load and increase life expectancy. For these regimens, a better toxicity profile is generally expected.

All chemotherapy regimens require that the patient be capable of undergoing the treatment. Performance status is often used as a measure to determine whether a patient can receive chemotherapy, or whether dose reduction is required.

B. Hormone Therapy

Several malignancies respond to hormonal therapy. Strictly speaking, this is not chemotherapy. Cancer arising from certain tissues, including the mammary and prostate glands, may be inhibited or stimulated by appropriate changes in hormone balance. Cancers that are most likely to be hormone-receptive include: Breast cancer, Prostate cancer, Ovarian cancer, and Endometrial cancer. Not every cancer of these types is hormone-sensitive, however. That is why the cancer must be analyzed to determine if hormone therapy is appropriate.

Breast cancer cells often highly express the estrogen and/or progesterone receptor. Inhibiting the production (with aromatase inhibitors) or action (with tamoxifen) of these hormones can often be used as an adjunct to therapy.

Hormone therapy may be used in combination with other types of cancer treatments, including surgery, radiation and chemotherapy. A hormone therapy can be used before a primary cancer treatment, such as before surgery to remove a tumor. This is called neoadjuvant therapy. Hormone therapy can sometimes shrink a tumor to a more manageable size so that it's easier to remove during surgery.

Hormone therapy is sometimes given in addition to the primary treatment—usually after—in an effort to prevent the cancer from recurring (adjuvant therapy). In some cases of advanced (metastatic) cancers, such as in advanced prostate cancer and advanced breast cancer, hormone therapy is sometimes used as a primary treatment.

The most common types of drugs for hormone-receptive cancers include: (1) Anti-hormones that block the cancer cell's ability to interact with the hormones that stimulate or support cancer growth. Though these drugs do not reduce the production of hormones, anti-hormones block the ability to use these hormones. Anti-hormones include the anti-estrogens tamoxifen (Nolvadex) and toremifene (Fareston) for breast cancer, and the anti-androgens flutamide (Eulexin) and bicalutamide (Casodex) for prostate cancer. (2) Aromatase inhibitors—Aromatase inhibitors (AIs) target enzymes that produce estrogen in postmenopausal women, thus reducing the amount of estrogen available to fuel tumors. AIs are only used in postmenopausal women because the drugs can't prevent the production of estrogen in women who haven't yet been through menopause. Approved AIs include letrozole (Femara), anastrozole (Arimidex) and exemestane (Aromasin). (3) Luteinizing hormone-releasing hormone (LH-RH) agonists and antagonists—LH-RH agonists—sometimes called analogs and LH-RH antagonists reduce the level of hormones by altering the mechanisms in the brain that tell the body to produce hormones. LH-RH agonists are essentially a chemical alternative to surgery for removal of the ovaries for women, or of the testicles for men. Depending on the cancer type, one might choose this route if they hope to have children in the future and want to avoid surgical castration. In most cases the effects of these drugs are reversible. Examples of LH-RH agonists include: Leuprolide (Lupron, Viadur, Eligard) for prostate cancer, Goserelin (Zoladex) for breast and prostate cancers, Triptorelin (Trelstar) for ovarian and prostate cancers and abarelix (Plenaxis).

One class of pharmaceuticals is the Selective Estrogen Receptor Modulators or SERMs. SERMs block the action of estrogen in the breast and certain other tissues by occupying estrogen receptors inside cells. SERMs include, but are not limited to tamoxifen (the brand name is Nolvadex, generic tamoxifen citrate); Raloxifene (brand name: Evista), and toremifene (brand name: Fareston).

### VI. Kits

Further embodiments of the invention include kits for the measurement, analysis, and reporting of gene expression and transcriptional output. A kit may include, but is not limited to microarray, quantitative RT-PCR, antibodies, labeling or other reagents and materials, as well as hardware and/or software for performing at least a portion of the methods described. For example, custom microarrays or analysis methods for existing microarrays are contemplated. Also, methods of the invention include methods of accessing and using a reporting system that compares a single result to a scale of clinical trial results. In yet still further aspects of the invention, a digital standard for data normalization is contemplated so that the assay result values from future samples would be able to be directly compared with the assay value results from past samples, such as from specific clinical trials.

### VII. Examples

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1

### Materials and Methods

Needle biopsy samples (fine needle aspirates—FNAs or core biopsies—CBX) were analyzed in order to examine genes correlated with the selected endpoint. The genes were identified by this method using these samples and methods to standardize data were done in order to facilitate calculation of the predictor indices consistently in different sample types such as biopsies, resected tissue from an excised tumor, and frozen tumor tissue.

Patients and Samples—

Patients prospectively consented to an Institutional Research Board approved research protocol (LAB99-402, USO-02-103, 2003-0321, I-SPY-1) to obtain a tumor biopsy by fine needle aspiration (FNA) or core biopsy (CBX) prior to any systemic therapy for genomic studies to develop and test predictors of treatment outcome. Clinical nodal status was determined before treatment from physical examination, with or without axillary ultrasound, with diagnostic FNA as required. Pathologic HER2 status was defined as negative according to the ASCO/CAP guidelines. Patients with any nuclear immunostaining for ER in the tumor cells were considered as eligible for adjuvant endocrine therapy. During this research, patients were consented to undergo pretreatment biopsy as fine needle aspiration (FNA) (Ayers, 2004; Hess, 2006) or core needle biopsy, of the primary breast tumor or ipsilateral axillary metastasis before starting chemotherapy as part of an ongoing pharmacogenomic marker discovery program. Gene expression data generated from the biopsies captures the molecular characteristics of the invasive cancer including the molecular class (Pusztai, 2003). At least 70% of all aspirations yielded at least 1 μg total RNA that is required for the gene expression profiling. The main reason for failure to obtain sufficient RNA was acellular aspirations. Three hundred and ten (310) patients with at least 1 μg RNA were included in this analysis. All patients received neoadjuvant chemotherapy consisting of a combination of either paclitaxel or docetaxel with anthracycline. At the completion of neoadjuvant chemotherapy all patients had modified radical mastectomy or lumpectomy and sentinel lymph node biopsy or axillary node dissection as determined appropriate by the surgeon. Patients who were ER-positive also received endocrine therapy as tamoxifen or aromatase inhibitor. Clinical characteristics of the patients are in Table 1A.

Discovery of Predictor of Relapse after Therapy:

Table 1B describes the breakdown of samples between FNAs and core biopsies and the treatments administered to the patients.

Validation of Predictors of Response and Relapse after Therapy:

Table 1A and 1B also describe the patients whose samples were used to validate the predictors developed for outcome of chemotherapy. Patient samples were collected at University of Texas M. D. Anderson Cancer Center (MDACC), LBJ Hospital, and US Oncology, in Houston, Tex. and at cancer centers in Peru, Mexico and Spain. During this research, patients were consented to undergo pretreatment biopsy as fine needle aspiration (FNA) (Ayers, 2004; Hess, 2006) or core needle biopsy, of the primary breast tumor or ipsilateral axillary metastasis before starting chemotherapy as part of an ongoing pharmacogenomic marker discovery program. One hundred and ninety eight (198) patients with at least 1 μg RNA and data on relapse-free survival to perform survival analysis were included in this analysis. All patients received either neoadjuvant chemotherapy, or in a small group, adjuvant chemotherapy, consisting of a combination of either paclitaxel or docetaxel with anthracycline. At the completion of neoadjuvant chemotherapy all patients had modified radical mastectomy or lumpectomy and sentinel lymph node biopsy or axillary node dissection as determined appropriate by the surgeon. Patients who were ER-positive also received endocrine therapy as tamoxifen or aromatase inhibitor. This study was approved by the institutional review boards (IRB) of the respective institutions and all patients signed an informed consent for voluntary participation.

TABLE 1A
Patient characteristics in development and validation of the predictors

| | Discovery Population | | | Validation Population | | | |
|---|---|---|---|---|---|---|---|
| | MDACC^{a} | I-SPY^{b} | Total | MDACC | LBJ^{c}/IN^{d}/GEI^{e} | USO^{f} | Total |
| Patients | 227 | 83 | 310 | 86 | 58 | 54 | 198 |
| Age | | | | | | | |
| <=50 | 112 (49%) | 30 (36%) | 142 (46%) | 48 (56%) | 30 (52%) | 31 (57%) | 109 (55%) |
| >50 | 115 (51%) | 53 (64%) | 168 (54%) | 38 (44%) | 28 (48%) | 23 (43%) | 89 (45%) |
| Mean (SD) | 51 (11) | 47 (8) | 50 (10) | 49 (11) | 51 (11) | 48 (9) | 49 (11) |
| Nodal status | | | | | | | |
| Pos | 165 (73%) | 58 (70%) | 223 (72%) | 52 (60%) | 42 (72%) | 34 (63%) | 128 (65%) |
| Neg | 62 (27%) | 25 (30%) | 87 (28%) | 34 (40%) | 16 (28%) | 20 (37%) | 70 (35%) |
| T stage | | | | | | | |
| 0 | 2 (1%) | 0 | 2 (1%) | 1 (1%) | 0 | 0 | 1 (1%) |
| 1 | 19 (8%) | 1 (1%) | 20 (6%) | 8 (9%) | 1 (1%) | 1 (2%) | 10 (5%) |
| 2 | 131 (58%) | 34 (41%) | 165 (53%) | 52 (61%) | 19 (33%) | 19 (35%) | 90 (45%) |
| 3 | 35 (15%) | 39 (47%) | 74 (24%) | 18 (21%) | 19 (33%) | 34 (63%) | 71 (36%) |
| 4 | 40 (18%) | 9 (11%) | 49 (16%) | 7 (8%) | 19 (33%) | 0 | 26 (13%) |
| Grade | | | | | | | |
| 1 | 13 (6%) | 6 (7%) | 19 (6%) | 7 (8%) | 5 (8%) | 1 (2%) | 13 (7%) |
| 2 | 92 (40%) | 25 (30%) | 117 (38%) | 28 (33%) | 19 (33%) | 16 (30%) | 63 (32%) |
| 3 | 122 (54%) | 29 (35%) | 151 (49%) | 51 (59%) | 23 (40%) | 34 (63%) | 108 (54%) |
| Unknown | 0 | 23 (28%) | 23 (7%) | 0 | 11 (19%) | 3 (5%) | 14 (7%) |
| AJCC^{g} | | | | | | | |
| Stage | | | | | | | |
| I | 6 (3%) | 0 | 6 (2%) | 2 (2%) | 0 | 0 | 2 (1%) |
| II | 126 (55%) | 39 (47%) | 165 (53%) | 57 66%) | 18 (31%) | 32 (59%) | 107 (54%) |
| III | 95 (42%) | 44 (53%) | 139 (45%) | 27 (32%) | 40 (69%) | 22 (41%) | 89 (45%) |
| ER^{h} Status | | | | | | | |
| Pos | 131 (58%) | 43 (52%) | 174 (56%) | 60 (70%) | 37 (64%) | 27 (50%) | 124 (63%) |
| Neg | 96 (42%) | 35 (42%) | 131 (42%) | 26 (30%) | 21 (36%) | 27 (50%) | 74 (37%) |
| Indeterminate | 0 | 5 (6%) | 5 (2%) | 0 | 0 | 0 | 0 |
| PRⁱ Status | | | | | | | |
| Pos | 102 (45%) | 40 (48%) | 142 (46%) | 43 (50%) | 31 (53%) | 28 (52%) | 102 (52%) |
| Neg | 125 (55%) | 37 (45%) | 162 (52%) | 43 (50%) | 27 (47%) | 26 (48%) | 96 (48%) |
| Indeterminate | 0 | 6 (7%) | 6 (2%) | 0 | 0 | 0 | 0 |

^{a}M.D. Anderson Cancer Center;
^{b}I-SPY-1 clinical trial;
^{c}Lyndon B. Johnson Hospital;
^{d}Instituto Nacional de Enfermedades Neoplásicas (INEN);
^{e}Grupo Español de Investigación en Cáncer de Mama (GEICAM);
^{f}US Oncology;
^{g}American Joint Committee on Cancer;
^{h}Estrogen receptor;
ⁱProgesterone receptor.

TABLE 1B
Chemotherapy And Pre-treatment Biopsy
Details for the Study Cohorts

| | Discovery | Validation |
|---|---|---|
| | Cohort | Cohort |
| | (N = 310) | (N = 198) |
| Needle Biopsy for Genomic Testing | | |
| FNA | 227 | 157 |
| CBX | 83 | 41 |
| Chemotherapy Regimen | | |
| Entirely Neoadjuvant | | |
| T × 12 → FAC × 4 → Sx¹ | 227 | 73 |
| AC × 4 → T/Tx × 4 → Sx² | 83 | — |
| TxX × 4 → FEC × 4 → Sx³ | — | 92 |
| Partial Neoadjuvant | | |
| FAC/FEC × 6 → Sx → T × 12⁴ | — | 18 |
| Entirely Adjuvant | | |
| Sx → T × 12 → FAC/FEC × 4⁵ | — | 12 |
| Sx → TxX × 4 → FEC × 4⁶ | — | 2 |
| Sx → Tx × 4 → FEC × 4⁷ | — | 1 |

FNA: fine needle aspiration
CBX: core needle biopsy
Sx: surgery
¹12 weekly doses of paclitaxel (T) followed by four cycles of fluorouracil (F), doxorubicin (A) and cyclophosphamide (C) and then surgery.
²Four cycles of doxorubicin (A) and cyclophosphamide (C) followed by four cycles of paclitaxel (T) (N = 60) or docetaxel (Tx) (N = 18) or taxane not specified (N = 5) and then surgery.
³Four cycles of docetaxel (Tx) with capecitabine (X) followed by four cycles of fluorouracil (F), epirubicin (E) and cyclophosphamide (C) and then surgery.
⁴Six cycles of fluorouracil (F), doxorubicin (A) or epirubicin (E), and cyclophosphamide (C) followed by surgery and then by 12 weekly doses of paclitaxel (T).
⁵Surgery followed by 12 weekly doses of paclitaxel (T) and then by four cycles of fluorouracil (F), doxorubicin (A) or epirubicin (E), and cyclophosphamide (C).
⁶Surgery followed by four cycles of docetaxel (Tx) with capecitabine (X) and then followed by four cycles of fluorouracil (F), epirubicin (E) and cyclophosphamide (C).
⁷Surgery followed by four cycles of docetaxel (Tx) and then by four cycles of fluorouracil (F), epirubicin (E) and cyclophosphamide (C).

RNA Extraction and Gene Expression Profiling—

Biopsy samples were either collected in 1.5 ml RNAlater™ (Qiagen, Valencia, Calif.) and stored locally at −70° C. and transported to the laboratory on dry ice (MDACC, INEN, LBJ, GEICAM) or couriered overnight in a cooler pack from clinics to the laboratory (USO), or were frozen, cryosectioned and an aliquot of RNA sent to the laboratory on dry ice (I-SPY). Details of our methods for RNA purification and microarray hybridization have been reported previously Rouzier, 2005; Stec, 2005; Symmans, 2003). Briefly, a single-round T7 amplification was used to generate biotin-labeled cRNA for hybridization to oligonucleotide microarrays (U133A GeneChip™, Affymetrix, Santa Clara, Calif.). Gene expression levels were derived from multiple oligonucleotide probes on the microarray that hybridize to different sequence sites of a gene transcript (probe sets).

Microarray Quality Control—

Quality control (QC) checks are performed at 3 levels (i) RNA yield, (ii) cRNA yield, and (ii) chip hybridization signal) and samples that fail at any level are not processed further. The amount and quality of RNA is assessed with NanoDrop ND-1000 Spectrophotometer (Thermo Fisher scientific In, Wilmington, Del., USA) and is generally considered adequate for further analysis if the OD 260/280 ratio is between 1.8-2.1 and the total RNA yield is ≧1.0 microgram. If total RNA yield is <1.0 microgram all remaining samples (if available) from that patient are used for RNA extraction. At least 10 μg of biotin-labeled cRNA need to be generated from a single-round in vitro transcription protocol to proceed with hybridized to U133A chips.

Microarray Data Normalization—

Raw intensity files (.CEL) from each microarray were processed using MAS5.0 (R/Bioconductor, www.bioconductor.org)¹ to normalize to a mean array intensity of 600 and to generate probe set-level expression values. Expression values were then log 2-transformed and subsequently scaled by the expression levels of 1322 breast cancer reference genes to reference values that had been established as the median expression of these genes in an independent reference cohort of invasive breast cancer (N=444). The quality of hybridization and microarray profiling was assessed based on a set of 8 metrics that compare the expression level of the reference genes in each sample to the historical reference values before and after scaling. Metrics include the median deviation, the inter-quartile range (IQR) of deviations, the Kolmogorov-Smirnov statistic for equality of the distributions and the p-value of the K-S statistic. Dimensionality was reduced through a principal component analysis (PCA) model of the 8 metrics which were further summarized in two multivariate statistics, the Hotteling T2 and the sum of squares of the residuals or Q statistic (Jackson & Mudholkar, 1979). Control limits for Q and T2 for sample acceptance were established from historical in-control samples. Prior to analysis for predictor development, 2,522 probe sets that either had low specificity (extensions_xfri_in their name), were housekeeping probes (starting with AFFX) or were not adequately expressed (log 2-transformed intensity of at least 5 in at least 75% of the arrays) were removed. A total of 16,289 probe sets (73% of all) were retained for further analysis.

### Example 2

### Predictor of Distant Relapse after Therapy or of Resistance to Therapy

Methods for Building Predictor of Survival Outcomes as a Result of Therapy—

Distant relapse-free survival (DRFS) was used as the endpoint of favorable outcome of therapy to build the predictor genes. Prior to analysis, probes that either had low specificity (those that include extensions_xfri_in their name) or housekeeping probes (those starting with AFFX) were selected and removed from the candidate probesets. This process removed 2522 probesets. Subsequently, a non-specific filter was applied to retain probesets that has log 2-transformed intensity of at least 5 in at least 75% of the arrays. A total of 16289 probesets (73% of all) were retained for further analysis.

The samples in the development cohort were subdivided in ER+ and ER− subsets and in lymph node negative (N0) and lymph positive (NP) subsets within each ER group. Means and standard deviations (SDs) of the 16289 genes were computed for each of the 4 subsets of cases. Within each ER cohort, the means and SDs for N0 and NP subsets were averaged to yield nodal-status adjusted statistics. These means and SDs were then used to scale the expression values of all probesets using the corresponding statistics for ER+ or ER− cases.

Each probeset was evaluated in a univariate Cox regression model for the significance of its association with risk of distant relapse. For this analysis, distant relapses or breast-cancer related deaths were considered as events, whereas local relapses were censored at the time of occurrence. Time to event was determined since the time of initial diagnosis. The significance of the association of each probeset to distant relapse risk was assessed based on the likelihood ratio test, which compares the log-likelihood of the model having the given probeset as the only covariate to the null model. The likelihood ratio statistic is distributed according to a chi-squared with one degree of freedom. P-values for the significance of each probeset were calculated from this distribution.

To account for sampling variability in the training dataset, Cox regression models for each probeset were fit repeatedly using a bootstrap procedure in which cases were sampled with replacement to generate bootstrapped datasets of the same size as the original dataset. This process was repeated 499 times, thus generating 500 estimates for the p-values of each probeset. The association of each probeset with distant relapse risk was assessed within each bootstrapped dataset at a critical significance level of 0.001 or 0.0005 to account for multiple testing. Those probesets that were called significant in at least 20% of the bootstrap replicates were selected as candidate probesets. This process was applied separately to the ER-positive and ER-negative cases in the training dataset and resulted in 235 and 268 candidate probesets in the ER+ and ER− subsets.

Final multivariate prediction models were built from the candidate probesets in the ER+ and ER− cohorts. Maximization of the partial likelihood associated with Cox proportional hazards models becomes problematic and non-unique if the number of covariates exceeds the number of available samples or if there is a high degree of colinearity between the predictors. To prevent this pathologic behavior, some sort of regularization or shrinkage needs to be applied to the regression coefficients to allow efficient estimation of the remaining ones. The Cox univariate shrinkage (CUS) approach was used for this purpose (Tibshirani, 2009), which is equivalent to the lasso estimate in standard regression analysis. The level of penalization is an adjustable parameter in the algorithm, with higher penalization resulting in smaller signatures. The optimal level of penalization was determined under 5-fold cross-validation as the penalization level that resulted in the shortest list of genes that yielded the highest incremental improvement in the Cox model's deviance.

The final predictors for ER+ and ER− subsets used 33 probesets and 27 probesets respectively to make the predictions. The probesets, genes that they encode for, and their weights (Cox coefficients) are shown in Table 2. The risk score is calculated by multiplying the scaled log 2-transformed expression level of each gene in a given sample by its corresponding weight and then adding up the weighted expression values for all genes in the signature. The following formula describes the score calculation for sample i:

$${y}_{i} = \{ \begin{matrix}{\sum }_{j = 1}^{{K}^{+}} {w}_{j}^{+} {z}_{ij}^{+} , & if ER positive \\ {\sum }_{j = 1}^{{K}^{-}} {w}_{j}^{-} {z}_{ij}^{-} , & if ER negative\end{matrix}$$

where wⱼ is the weight of gene j in the signature, zᵢⱼ is the log 2-transformed and scaled expression value of gene j in sample i, K is the number of genes in the signature, and the + or − symbols refer to the ER+ and ER− signatures.

A cut point was selected to dichotomize the risk score and predict two risk classes. The optimal cutoff was selected in order to maximize the accuracy of the prediction of 5-yr distant relapse outcome by the risk classes. A cutoff of 0 was selected for both the ER+ and ER− scores. Positive scores signify “High risk” class, i.e. higher risk of distant relapse and a zero or negative score signifies “Low risk”.

TABLE 2
Genes used for prediction of distant relapse risk in ER-stratified patient subsets

| | | | | | | |
|---|---|---|---|---|---|---|
| Probe Set | Symbol | Description | GeneID | Chromosome | Cytoband | Weight |

ER-Positive

| | | | | | | | |
|---|---|---|---|---|---|---|---|
| 1 | 212174_at | AK2 | adenylate kinase 2 | 204 | 1 | 1p34 | 0.0011 |
| 2 | 215407_s_at | ASTN2 | astrotactin 2 | 23245 | 9 | 9q33.1 | −0.0131 |
| 3 | 205626_s_at | CALB1 | calbindin 1, 28 kDa | 793 | 8 | 8q21.3-q22.1 | 0.008 |
| 4 | 212816_s_at | CBS | cystathionine-beta- | 875 | 21 | 21q22.3 | 0.0116 |
| | | | synthase | | | | |
| 5 | 216923_at | CDLK5 | cyclin-dependent kinase | 6792 | X | Xp22.13 | −0.0084 |
| | | | like 5 | | | | |
| 6 | 205471_s_at | DACH1 | dachshund homolog 1 | 1602 | 13 | 13q22 | −0.0043 |
| | | | (Drosophila) | | | | |
| 7 | 221681_s_at | DSPP | dentin | 1834 | 4 | 4q21.3 | −0.0285 |
| | | | sialophosphoprotein | | | | |
| 8 | 201539_s_at | FHL1 | four and a half LIM | 2273 | X | Xq26 | 0.0142 |
| | | | domains 1 | | | | |
| 9 | 215744_at | FUS | fusion (involved in t(12; 16) | 2521 | 16 | 16p11.2 | −0.0016 |
| | | | in malignant liposarcoma) | | | | |
| 10 | 209604_s_at | GATA3 | GATA binding protein 3 | 2625 | 10 | 10p15 | −0.0414 |
| 11 | 209602_s_at | GATA3 | GATA binding protein 3 | 2625 | 10 | 10p15 | −0.0285 |
| 12 | 209603_at | GATA3 | GATA binding protein 3 | 2625 | 10 | 10p15 | −0.0067 |
| 13 | 203821_at | HBEGF | heparin-binding EGF-like | 1839 | 5 | 5q23 | −0.0126 |
| | | | growth factor | | | | |
| 14 | 219976_at | HOOK1 | hook homolog 1 | 51361 | 1 | 1p32.1 | −0.0136 |
| | | | (Drosophila) | | | | |
| 15 | 212531_at | LCN2 | lipocalin 2 | 3934 | 9 | 9q34 | 0.0411 |
| 16 | 220906_at | LDB2 | LIM domain binding 2 | 9079 | 4 | p15.32 | −0.0358 |
| 17 | 217506_at | LOC339290 | hypothetical LOC339290 | 339290 | 18 | 18p11.31 | −0.0171 |
| 18 | 204058_at | ME1 | malic enzyme 1, | 4199 | 6 | 6q12 | 0.0002 |
| | | | NADP(+)-dependent, | | | | |
| | | | cytosolic | | | | |
| 19 | 200899_s_at | MGEA5 | meningioma expressed | 10724 | 10 | 10q24.1-q24.3 | −0.0023 |
| | | | antigen 5 (hyaluronidase) | | | | |
| 20 | 203419_at | MLL4 | myeloid/lymphoid or | 9757 | 19 | 19q13.1 | −0.0097 |
| | | | mixed-lineage leukemia 4 | | | | |
| 21 | 211874_s_at | MYST4 | MYST histone | 23522 | 10 | 10q22.2 | −0.0336 |
| | | | acetyltransferase | | | | |
| | | | (monocytic leukemia) 4 | | | | |
| 22 | 40569_at | MZF1 | myeloid zinc finger 1 | 7593 | 19 | 19q13.4 | −0.0349 |
| 23 | 203621_at | NDUFB5 | NADH dehydrogenase | 4711 | 3 | 3q26.33 | 0.0448 |
| | | | (ubiquinone) 1 beta | | | | |
| | | | subcomplex, 5, 16 kDa | | | | |
| 24 | 202886_s_at | PPP2R1B | protein phosphatase 2 | 5519 | 11 | 11q23.2 | 0.0061 |
| | | | (formerly 2A), regulatory | | | | |
| | | | subunit A, beta isoform | | | | |
| 25 | 201834_at | PRKAB1 | protein kinase, AMP- | 5564 | 12 | 12q24.1 | −0.0341 |
| | | | activated, beta 1 non- | | | | |
| | | | catalytic subunit | | | | |
| 26 | 212743_at | RCHY1 | ring finger and CHY zinc | 25898 | 4 | 4q21.1 | −0.0127 |
| | | | finger domain containing 1 | | | | |
| 27 | 219869_s_at | SLC39A8 | solute carrier family 39 | 64116 | 4 | 4q22-q24 | 0.0262 |
| | | | (zinc transporter), member 8 | | | | |
| 28 | 210692_s_at | SLC43A3 | solute carrier family 43, | 29015 | 11 | 11q11 | 0.0075 |
| | | | member 3 | | | | |
| 29 | 213103_at | STARD13 | StAR-related lipid transfer | 90627 | 13 | 13q12-q13 | −0.0185 |
| | | | (START) domain | | | | |
| | | | containing 13 | | | | |
| 30 | 202342_s_at | TRIM2 | tripartite motif-containing 2 | 23321 | 4 | 4q31.3 | 0.0088 |
| 31 | 212534_at | ZNF24 | zinc finger protein 24 | 7572 | 18 | 18q12 | −0.0025 |
| 32 | 219635_at | ZNF606 | zinc finger protein 606 | 80095 | 19 | 19q13.4 | −0.0198 |
| 33 | 214202_at | — | — | — | 5 | 5q22.3 | −0.0421 |

ER-Negative

| | | | | | | | |
|---|---|---|---|---|---|---|---|
| 1 | 200982_s_at | ANXA6 | annexin A6 | 309 | 5 | 5q32-q34 | 0.0136 |
| 2 | 212136_at | ATP2B4 | ATPase, Ca++ transporting, plasma membrane 4 | 493 | 1 | 1q32.1 | 0.0123 |
| 3 | 205379_at | CBR3 | carbonyl reductase 3 | 874 | 21 | 21q22.2 | −0.0067 |
| 4 | 219755_at | CBX8 | chromobox homolog 8 (Pc class homolog, | 57332 | 17 | 17q25.3 | −0.0023 |
| | | | Drosophila) | | | | |
| 5 | 204720_s_at | DNAJC6 | DnaJ (Hsp40) homolog, subfamily C, member 6 | 9829 | 1 | 1pter-q31.3 | 0.0022 |
| 6 | 203303_at | DYNLT3 | dynein, light chain, Tctex-type 3 | 6990 | X | Xp21 | 0.0041 |
| 7 | 216682_s_at | FAM48A | family with sequence similarity 48, member A | 55578 | 13 | 13q13.3 | 0.0044 |
| 8 | 206847_s_at | HOXA7 | homeobox A7 | 3204 | 7 | 7p15-p14 | −0.0323 |
| 9 | 219284_at | HSPBAP1 | HSPB (heat shock 27 kDa) associated protein 1 | 79663 | 3 | 3q21.1 | 0.0068 |
| 10 | 210036_s_at | KCNH2 | potassium voltage-gated channel, subfamily H | 3757 | 7 | 7q35-q36 | 0.0158 |
| | | | (eag-related), member 2 | | | | |
| 11 | 217929_s_at | KIAA0319L | KIAA0319-like | 79932 | 1 | 1p34.2 | −0.0044 |
| 12 | 201932_at | LRRC41 | leucine rich repeat containing 41 | 10489 | 1 | 1p34.1 | −0.0089 |
| 13 | 205301_s_at | OGG1 | 8-oxoguanine DNA glycosylase | 4968 | 3 | 3p26.2 | −0.0025 |
| 14 | 208393_s_at | RAD50 | RAD50 homolog (S. cerevisiae) | 10111 | 5 | 5q31 | 0.0404 |
| 15 | 203286_at | RNF44 | ring finger protein 44 | 22838 | 5 | 5q35.2 | 0.0061 |
| 16 | 213044_at | ROCK1 | Rho-associated, coiled-coil containing protein | 6093 | 18 | 18q11.1 | 0.0138 |
| | | | kinase 1 | | | | |
| 17 | 203889_at | SCG5 | secretogranin V (7B2 protein) | 6447 | 15 | 15q13-q14 | 0.0065 |
| 18 | 221053_s_at | TDRKH | tudor and KH domain containing | 11022 | 1 | 1q21 | 0.0107 |
| 19 | 203254_s_at | TLN1 | talin 1 | 7094 | 9 | 9p13 | 0.0167 |
| 20 | 210180_s_at | TRA2B | transformer 2 beta homolog (Drosophila) | 6434 | 3 | 3q26.2-q27 | −0.001 |
| 21 | 221836_s_at | TRAPPC9 | trafficking protein particle complex 9 | 83696 | 8 | 8q24.3 | 0.0165 |
| 22 | 208349_at | TRPA1 | transient receptor potential cation channel, | 8989 | 8 | 8q13 | 0.025 |
| | | | subfamily A, member 1 | | | | |
| 23 | 216374_at | TSPY1 | testis specific protein, Y-linked 1 | 7258 | Y | Yp11.2 | 0.0079 |
| 24 | 218715_at | UTP6 | UTP6, small subunit (SSU) processome | 55813 | 17 | 17q112 | 0.0142 |
| | | | component, homolog (yeast) | | | | |
| 25 | 208453_s_at | XPNPEP1 | X-prolyl aminopeptidase (aminopeptidase P) 1, | 7511 | 10 | 10q25.3 | −0.0208 |
| | | | soluble | | | | |
| 26 | 214900_at | ZKSCAN1 | zinc finger with KRAB and SCAN domains 1 | 7586 | 7 | 7q21.3-q22.1 | −0.0032 |
| 27 | 215298_at | — | — | — | 4 | 4q8.3 | 0.0039 |

### Example 3

### Performance of Relapse-Based Predictor in Chemotherapy Outcomes Prediction

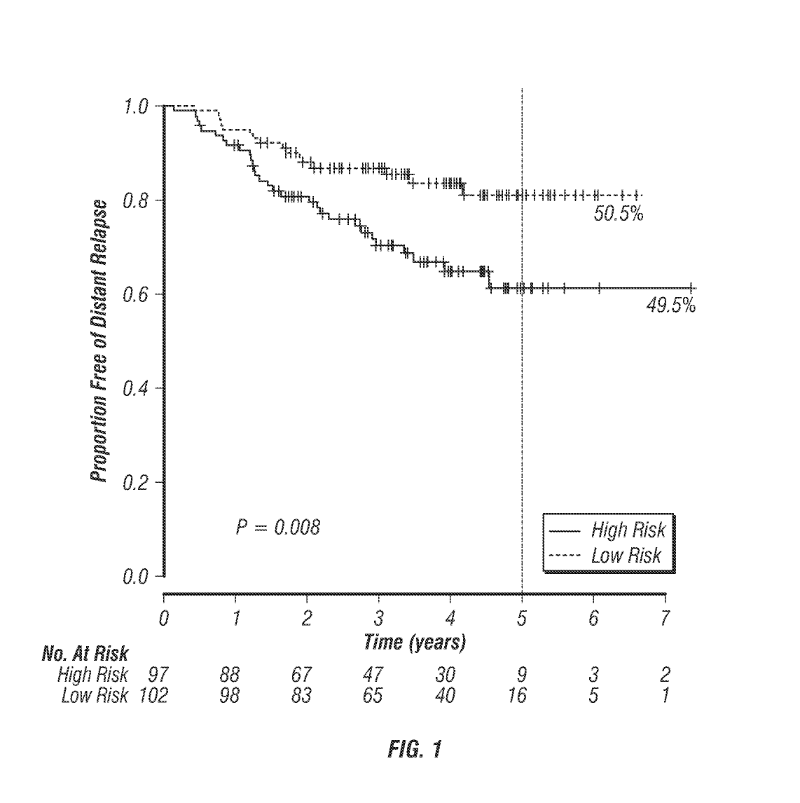

FIG. 1 shows the survival outcome of patients from the validation cohort (Table 1A) predicted as good and poor responders by the ER-stratified outcomes predictor described in Example 2. Survival is defined by distant relapse-free survival (DRFS) over a period of about 60 months since the initial biopsy. These patients have undergone surgery where it was considered appropriate and the ER-positive patients received hormonal therapy (tamoxifen or aromatase inhibitor) for 5 years after the surgery. ER-negative patients did not receive any additional treatment post-surgery.

The plot shows that predicted good and poor responders to taxane-chemotherapy (FIG. 1) have distinctly separated relapse-free survival curves (p=0.008). The good responders (51%) or “low-risk” patients show a fewer number of distant relapse events (˜85% relapse-free after 60 months) whereas the remaining patients show considerably higher relapse rates among the patients (˜60% DRFS after 60 months).

### Example 4

### Predictor of Response to Chemotherapy

Patients and Samples—

Patient samples used were those shown in Table 1A. All other laboratory analytic methods were the same as in Example 1.

Methods for Building Predictors of Response to Chemotherapy—

The inventors used the response endpoint RCB0/I, representing no residual disease or minimal residual disease measured at the completion of neoadjuvant chemotherapy, to identify genes that differentiated patients who responded to chemotherapy versus all others in the discovery cohort of Table 1A. Prior to analysis, probes that either had low specificity (those that include extensions_xfri_in their name) or housekeeping probes (those starting with AFFX) were selected and removed from the candidate probesets. This process removed 2522 probesets. Subsequently, a non-specific filter was applied to retain probesets that has log 2-transformed intensity of at least 5 in at least 75% of the arrays. A total of 16289 probesets (73% of all) were retained for further analysis.

The samples in the development cohort were subdivided in ER+ and ER− subsets and in lymph node negative (N0) and lymph positive (NP) subsets within each ER group. Means and standard deviations (SDs) of the 16289 genes were computed for each of the 4 subsets of cases. Within each ER cohort, the means and SDs for N0 and NP subsets were averaged to yield nodal-status adjusted statistics. These means and SDs were then used to scale the expression values of all probesets using the corresponding statistics for ER+ or ER− cases.

Each probeset was evaluated for differential expression in the two responder groups (RCB-0/I vs rest) using an unequal variance t-statistic based on the trimmed means and trimmed standard deviations in the two groups using a trim fraction of 0.025 (i.e. the lowest 2.5% and highest 2.5% values were eliminated and the statistics were calculated on the remaining 95% of the observations in each group). Degrees of freedom for the unequal variance t-statistic were estimated based on Satterthwaite's approximation (Armitage, Berry & Matthews, 2002). The significance of association of each probe set with response was assessed based on the unequal variance t-statistic. P-values for the significance of each probeset were calculated from the t-distribution with the corresponding degrees of freedom.

To account for sampling variability in the training dataset, the differential expression analysis for each probeset described in the previous paragraph was performed repeatedly using a bootstrap procedure in which cases were sampled with replacement to generate bootstrapped datasets of the same size as the original dataset. This process was repeated 499 times, thus generating 500 estimates for the p-values of each probeset. The association of each probeset with distant relapse risk was assessed within each bootstrapped dataset at a critical significance level of 0.0005 to account for multiple testing. Those probesets that were called significant in at least 30% of the bootstrap replicates were selected as candidate probesets. This process was applied separately to the ER-positive and ER-negative cases in the training dataset and resulted in 209 and 244 candidate probesets in the ER+ and ER-subsets.

In developing the RCB-based chemothereapy response predictor, the inventors used an approach that combines feature selection and model discovery using a multivariate penalized approach called Gradient Directed Regularization developed by Prof. J. Friedman at Stanford University, a description of which can be found on the World Wide Web at stat.stanford.edu/˜jhf/ftp/pathlite.pdf. The informative genes are selected through penalization using the maximization of the area under the ROC curve (AUC) as the optimization criterion. Ma and Huang have previously used a similar approach for disease classification (Ma, 2006).

For predictor discovery and evaluation the inventors followed a cross-validation protocol. First, the input dataset is randomly partitioned into a training set and a test set. A 5-fold cross-validation for a 4:1 split stratified by response group between training and test sets was used (Dudoit, 2002). The training set consisting of ⅘ of the original data is used to develop the predictor. The algorithm starts with the same initial list of candidate genes that were determined through the bootstrap procedure and iteratively refines the predictor by selecting genes that contribute in maximizing the AUC of the candidate predictor. The maximum level of penalization is used to derive the most parsimonious predictors. Since different optimal reporter gene sets might result from the different internal cross-validation folds, the number of times each gene is selected is tracked to provide a measure of its importance or its reliability. The trained predictor is then tested on the ⅕ hold-out part of the training dataset and its performance is evaluated based on the AUC.

The entire process of randomly splitting the data to a training- and a test-set was repeated 499 times to obtain the distributions and summary statistics of the performance metrics from the cross-validated replicates.

The final predictors for ER+ and ER− subsets used 39 probesets and 55 probesets respectively to make the predictions. The probesets, genes that they encode for, and their weights (coefficients) are shown in Table 3. The risk score is calculated by multiplying the scaled log 2-transformed expression level of each gene in a given sample by its corresponding weight and then adding up the weighted expression values for all genes in the signature. The following formula describes the score calculation for sample i:

$${y}_{i} = \{ \begin{matrix}{\sum }_{j = 1}^{{K}^{+}} {w}_{j}^{+} {z}_{ij}^{+} , & if ER positive \\ {\sum }_{j = 1}^{{K}^{-}} {w}_{j}^{-} {z}_{ij}^{-} , & if ER negative\end{matrix}$$

where wⱼ is the weight of gene j in the signature, zᵢⱼ is the log 2-transformed and scaled expression value of gene j in sample i, K is the number of genes in the signature, and the + or − symbols refer to the ER+ and ER− signatures.

A cut point was selected to dichotomize the risk score and predict two risk classes. The optimal cutoff was selected in order to maximize the accuracy of the prediction. A cutoff of 0 was selected for both the ER+ and ER− scores. Positive scores signify “responders” and a zero or negative score signifies “non-responders”.

TABLE 3
Genes used for prediction of response, RCB-0/I, in ER-stratified patient subsets

| | | | | | | |
|---|---|---|---|---|---|---|
| Probe Set | Symbol | Description | GeneID | Chromosome | Cytoband | Weight |

ER-Positive

| | | | | | | | |
|---|---|---|---|---|---|---|---|
| 1 | 204332_s_at | AGA | aspartylglucosaminidase | 175 | 4 | 4q32-q33 | 1.023626 |
| 2 | 36865_at | ANGEL1 | angel homolog 1 | 23357 | 14 | 14q24.3 | 0.538063 |
| | | | (Drosophila) | | | | |
| 3 | 219437_s_at | ANKRD11 | ankyrin repeat domain | 29123 | 16 | 16q24.3 | 0.26952 |
| | | | 11 | | | | |
| 4 | 205865_at | ARID3A | AT rich interactive | 1820 | 19 | 19p13.3 | 0.832093 |
| | | | domain 3A (BRIGHT- | | | | |
| | | | like) | | | | |
| 5 | 215407_s_at | ASTN2 | astrotactin 2 | 23245 | 9 | 9q33.1 | 1.081851 |
| 6 | 204493_at | BID | BH3 interacting domain | 637 | 22 | 22q11.1 | 0.351295 |
| | | | death agonist | | | | |
| 7 | 205557_at | BPI | bactericidal/permeability- | 671 | 20 | 20q11.23-q12 | −1.05657 |
| | | | increasing protein | | | | |
| 8 | 42361_g_at | CCHCR1 | coiled-coil alpha-helical | 54535 | 6 | 6p21.3 | −0.19308 |
| | | | rod protein 1 | | | | |
| 9 | 205937_at | CGREF1 | cell growth regulator | 10669 | 2 | 2p23.3 | 0.616448 |
| | | | with EF-hand domain 1 | | | | |
| 10 | 208817_at | COMT | catechol-O- | 1312 | 22 | 22q11.21 | 0.964167 |
| | | | methyltransferase | | | | |
| 11 | 202250_s_at | DCAF8 | DDB1 and CUL4 | 50717 | 1 | 1q22-q23 | 0.438059 |
| | | | associated factor 8 | | | | |
| 12 | 202570_s_at | DLGAP4 | discs, large (Drosophila) | 22839 | 20 | 20q11.23 | −0.03735 |
| | | | homolog-associated | | | | |
| | | | protein 4 | | | | |
| 13 | 218103_at | FTSJ3 | FtsJ homolog 3 (E. coli) | 117246 | 17 | 17q23.3 | 0.902969 |
| 14 | 216651_s_at | GAD2 | glutamate | 2572 | 10 | 10p11.23 | 1.191928 |
| | | | decarboxylase 2 | | | | |
| | | | (pancreatic islets and | | | | |
| | | | brain, 65 kDa) | | | | |
| 15 | 205505_at | GCNT1 | glucosaminyl (N-acetyl) | 2650 | 9 | 9q13 | 0.635989 |
| | | | transferase 1, core 2 | | | | |
| | | | (beta-1,6-N- | | | | |
| | | | acetylglucosaminyltransferase) | | | | |
| 16 | 213020_at | GOSR1 | golgi SNAP receptor | 9527 | 17 | 17q11 | 0.041002 |
| | | | complex member 1 | | | | |
| 17 | 212597_s_at | HMGXB4 | HMG box domain | 10042 | 22 | 22q13.1 | 0.241141 |
| | | | containing 4 | | | | |
| 18 | 212898_at | KIAA0406 | KIAA0406 | 9675 | 20 | 20q11.23 | −0.37731 |
| 19 | 220652_at | KIF24 | kinesin family member | 347240 | 9 | 9p13.3 | −0.85991 |
| | | | 24 | | | | |
| 20 | 218486_at | KLF11 | Kruppel-like factor 11 | 8462 | 2 | 2p25 | 0.145703 |
| 21 | 202057_at | KPNA1 | karyopherin alpha 1 | 3836 | 3 | 3q21 | 0.047619 |
| | | | (importin alpha 5) | | | | |
| 22 | 209204_at | LMO4 | LIM domain only 4 | 8543 | 1 | 1p22.3 | 0.906757 |
| 23 | 201818_at | LPCAT1 | lysophosphatidylcholine | 79888 | 5 | 5p15.33 | 0.602505 |
| | | | acyltransferase 1 | | | | |
| 24 | 208328_s_at | MEF2A | myocyte enhancer | 4205 | 15 | 15q26 | 0.196532 |
| | | | factor 2A | | | | |
| 25 | 215491_at | MYCL1 | v-myc | 4610 | 1 | 1p34.2 | 1.199616 |
| | | | myelocytomatosis viral | | | | |
| | | | oncogene homolog 1, | | | | |
| | | | lung carcinoma derived | | | | |
| | | | (avian) | | | | |
| 26 | 202944_at | NAGA | N- | 4668 | 22 | 22q11 | 0.053596 |
| | | | acetylgalactosaminidase, | | | | |
| | | | alpha- | | | | |
| 27 | 218886_at | PAK1IP1 | PAK1 interacting protein 1 | 55003 | 6 | 6p24.2 | −0.39992 |
| 28 | 207081_s_at | PI4KA | phosphatidylinositol 4- | 5297 | 22 | 22q11.21 | 0.879705 |
| | | | kinase, catalytic, alpha | | | | |
| 29 | 210771_at | PPARA | peroxisome proliferator- | 5465 | 22 | 22q12-q13.1 | 0.771244 |
| | | | activated receptor alpha | | | | |
| 30 | 203096_s_at | RAPGEF2 | Rap guanine nucleotide | 9693 | 4 | 4q32.1 | 0.645585 |
| | | | exchange factor (GEF) 2 | | | | |
| 31 | 218593_at | RBM28 | RNA binding motif | 55131 | 7 | 7q32.1 | 0.533325 |
| | | | protein 28 | | | | |
| 32 | 211678_s_at | RNF114 | ring finger protein 114 | 55905 | 20 | 20q13.13 | 1.178185 |
| 33 | 202762_at | ROCK2 | Rho-associated, coiled- | 9475 | 2 | 2p24 | 1 |
| | | | coil containing protein | | | | |
| | | | kinase 2 | | | | |
| 34 | 206239_s_at | SPINK1 | serine peptidase | 6690 | 5 | 5q32 | 0.620242 |
| | | | inhibitor, Kazal type 1 | | | | |
| 35 | 221276_s_at | SYNC | syncoilin, intermediate | 81493 | 1 | 1p34.3-p33 | −0.38482 |
| | | | filament protein | | | | |
| 36 | 213155_at | WSCD1 | WSC domain containing 1 | 23302 | 17 | 17p13.2 | −0.31573 |
| 37 | 37117_at | PRR5 | proline rich 5 (renal) | 55615 | 22 | 22q13 | 0.106363 |
| 38 | 220855_at | AC091271.1 | no-protein transcript | — | 17 | 17q23.2 | −0.3595 |
| 39 | 222275_at | — | — | — | 5 | 5p12 | 0.03155 |

ER-Negative

| | | | | | | | |
|---|---|---|---|---|---|---|---|
| 1 | 202442_at | AP3S1 | adaptor-related protein | 1176 | 5 | 5q22 | −0.19044 |
| | | | complex 3, sigma 1 | | | | |
| | | | subunit | | | | |
| 2 | 212135_s_at | ATP2B4 | ATPase, Ca++ | 493 | 1 | 1q32.1 | −0.3245 |
| | | | transporting, plasma | | | | |
| | | | membrane 4 | | | | |
| 3 | 217911_s_at | BAG3 | BCL2-associated | 9531 | 10 | 10q25.2-q26.2 | −0.23225 |
| | | | athanogene 3 | | | | |
| 4 | 210214_s_at | BMPR2 | bone morphogenetic | 659 | 2 | 2q33-q34 | 0.814841 |
| | | | protein receptor, type II | | | | |
| | | | (serine/threonine kinase) | | | | |
| 5 | 202048_s_at | CBX6 | chromobox homolog 6 | 23466 | 22 | 22q13.1 | −1.02907 |
| 6 | 203653_s_at | COIL | coilin | 8161 | 17 | 17q22-q23 | 0.078687 |
| 7 | 203633_at | CPT1A | carnitine | 1374 | 11 | 11q13.1-q13.2 | −0.06407 |
| | | | palmitoyltransferase 1A | | | | |
| | | | (liver) | | | | |
| 8 | 210096_at | CYP4B1 | cytochrome P450, family | 1580 | 1 | 1p34-p12 | −0.39651 |
| | | | 4, subfamily B, | | | | |
| | | | polypeptide 1 | | | | |
| 9 | 212838_at | DNMBP | dynamin binding protein | 23268 | 10 | 10q24.2 | −0.07158 |
| 10 | 219850_s_at | EHF | ets homologous factor | 26298 | 11 | 11p12 | 0.115972 |
| 11 | 201936_s_at | EIF4G3 | eukaryotic translation | 8672 | 1 | 1p36.12 | −0.08341 |
| | | | initiation factor 4 gamma, 3 | | | | |
| 12 | 217254_s_at | EPO | erythropoietin | 2056 | 7 | 7q22 | −0.9403 |
| 13 | 205774_at | F12 | coagulation factor XII | 2161 | 5 | 5q33-qter | −0.21253 |
| | | | (Hageman factor) | | | | |
| 14 | 218532_s_at | FAM134B | family with sequence | 54463 | 5 | 5p15.1 | −0.12462 |
| | | | similarity 134, member B | | | | |
| 15 | 200709_at | FKBP1A | FK506 binding protein | 2280 | 20 | 20p13 | −0.06741 |
| | | | 1A, 12 kDa | | | | |
| 16 | 212294_at | GNG12 | guanine nucleotide | 55970 | 1 | 1p31.3 | −0.2595 |
| | | | binding protein (G | | | | |
| | | | protein), gamma 12 | | | | |
| 17 | 211525_s_at | GP5 | glycoprotein V (platelet) | 2814 | 3 | 3q29 | −0.52858 |
| 18 | 212090_at | GRINA | glutamate receptor, | 2907 | 8 | 8q24.3 | −0.02213 |
| | | | ionotropic, N-methyl D- | | | | |
| | | | aspartate-associated | | | | |
| | | | protein 1 (glutamate | | | | |
| | | | binding) | | | | |
| 19 | 213053_at | HAUS5 | HAUS augmin-like | 23354 | 19 | 19q13.12 | 0.395212 |
| | | | complex, subunit 5 | | | | |
| 20 | 214537_at | HIST1H1D | histone cluster 1, H1d | 3007 | 6 | 6p21.3 | 0.029003 |
| 21 | 206194_at | HOXC4 | homeobox C4 | 3221 | 12 | 12q13.3 | −0.10183 |
| 22 | 204544_at | HPS5 | Hermansky-Pudlak | 11234 | 11 | 11p14 | 0.203156 |
| | | | syndrome 5 | | | | |
| 23 | 205700_at | HSD17B6 | hydroxysteroid (17-beta) | 8630 | 12 | 12q13 | −0.88741 |
| | | | dehydrogenase 6 | | | | |
| | | | homolog (mouse) | | | | |
| 24 | 209575_at | IL10RB | interleukin 10 receptor, | 3588 | 21 | 21q22.1-q22.2 | 0.162807 |
| | | | beta | | | | |
| 25 | 215177_s_at | ITGA6 | integrin, alpha 6 | 3655 | 2 | 2q31.1 | 0.34206 |
| 26 | 221986_s_at | KLHL24 | kelch-like 24 (Drosophila) | 54800 | 3 | 3q27.1 | 1 |
| 27 | 208107_s_at | LOC81691 | exonuclease NEF-sp | 81691 | 16 | 16p12.3 | 0.618062 |
| 28 | 221650_s_at | MED18 | mediator complex | 54797 | 1 | 1p35.3 | −0.05596 |
| | | | subunit 18 | | | | |
| 29 | 218251_at | MID1IP1 | MID1 interacting protein | 58526 | X | Xp11.4 | −0.41753 |
| | | | 1 (gastrulation specific | | | | |
| | | | G12 homolog | | | | |
| | | | (zebrafish)) | | | | |
| 30 | 215563_s_at | MSTP9 | macrophage stimulating, | 11223 | 1 | 1p36.13 | −0.02463 |
| | | | pseudogene 9 | | | | |
| 31 | 221207_s_at | NBEA | neurobeachin | 26960 | 13 | 13q13 | −0.45289 |
| 32 | 208926_at | NEU1 | sialidase 1 (lysosomal | 4758 | 6 | 6p21.3 | 0.27621 |
| | | | sialidase) | | | | |
| 33 | 204107_at | NFYA | nuclear transcription | 4800 | 6 | 6p21.3 | −0.10057 |
| | | | factor Y, alpha | | | | |
| 34 | 218410_s_at | PGP | phosphoglycolate | 283871 | 16 | 16p13.3 | −0.13051 |
| | | | phosphatase | | | | |
| 35 | 211159_s_at | PPP2R5D | protein phosphatase 2, | 5528 | 6 | 6p21.1 | 0.218826 |
| | | | regulatory subunit B′, | | | | |
| | | | delta isoform | | | | |
| 36 | 205617_at | PRRG2 | proline rich Gla (G- | 5639 | 19 | 19q13.33 | 0.752739 |
| | | | carboxyglutamic acid) 2 | | | | |
| 37 | 203038_at | PTPRK | protein tyrosine | 5796 | 6 | 6q22.2-q22.3 | 0.268374 |
| | | | phosphatase, receptor | | | | |
| | | | type, K | | | | |
| 38 | 203831_at | R3HDM2 | R3H domain containing 2 | 22864 | 12 | 12q13.3 | −0.04695 |
| 39 | 201779_s_at | RNF13 | ring finger protein 13 | 11342 | 3 | 3q25.1 | 0.247392 |
| 40 | 203286_at | RNF44 | ring finger protein 44 | 22838 | 5 | 5q35.2 | −0.07864 |
| 41 | 221524_s_at | RRAGD | Ras-related GTP binding D | 58528 | 6 | 6q15-q16 | 0.616503 |
| 42 | 212416_at | SCAMP1 | secretory carrier | 9522 | 5 | 5q13.3-q14.1 | −0.96624 |
| | | | membrane protein 1 | | | | |
| 43 | 207707_s_at | SEC13 | SEC13 homolog (S. cerevisiae) | 6396 | 3 | 3p25-p24 | 0.706684 |
| 44 | 201915_at | SEC63 | SEC63 homolog (S. cerevisiae) | 11231 | 6 | 6q21 | 0.383853 |
| 45 | 203580_s_at | SLC7A6 | solute carrier family 7 | 9057 | 16 | 16q22.1 | −0.16415 |
| | | | (cationic amino acid | | | | |
| | | | transporter, y+ system), | | | | |
| | | | member 6 | | | | |
| 46 | 212257_s_at | SMARCA2 | SWI/SNF related, matrix | 6595 | 9 | 9p22.3 | 0.152197 |
| | | | associated, actin | | | | |
| | | | dependent regulator of | | | | |
| | | | chromatin, subfamily a, | | | | |
| | | | member 2 | | | | |
| 47 | 201794_s_at | SMG7 | Smg-7 homolog, | 9887 | 1 | 1q25 | −0.33961 |
| | | | nonsense mediated | | | | |
| | | | mRNA decay factor (C. elegans) | | | | |
| 48 | 202991_at | STARD3 | StAR-related lipid | 10948 | 17 | 17q11-q12 | 0.579916 |
| | | | transfer (START) domain | | | | |
| | | | containing 3 | | | | |
| 49 | 210294_at | TAPBP | TAP binding protein | 6892 | 6 | 6p21.3 | 0.04522 |
| | | | (tapasin) | | | | |
| 50 | 217711_at | TEK | TEK tyrosine kinase, | 7010 | 9 | 9p21 | −0.06112 |
| | | | endothelial | | | | |
| 51 | 212638_s_at | WWP1 | WW domain containing | 11059 | 8 | 8q21 | −0.37266 |
| | | | E3 ubiquitin protein | | | | |
| | | | ligase 1 | | | | |
| 52 | 213081_at | ZBTB22 | zinc finger and BTB | 9278 | 6 | 6p21.3 | −0.16771 |
| | | | domain containing 22 | | | | |
| 53 | 216738_at | — | — | — | 3 | 3p25.3 | −0.10674 |
| 54 | 220820_at | — | — | — | 10 | 10q11.23 | −0.3542 |
| 55 | 222312_s_at | — | — | — | 1 | 1p22.3 | −0.11559 |

### Example 5

### Performance of Response-Based Predictor in Validation Cohort

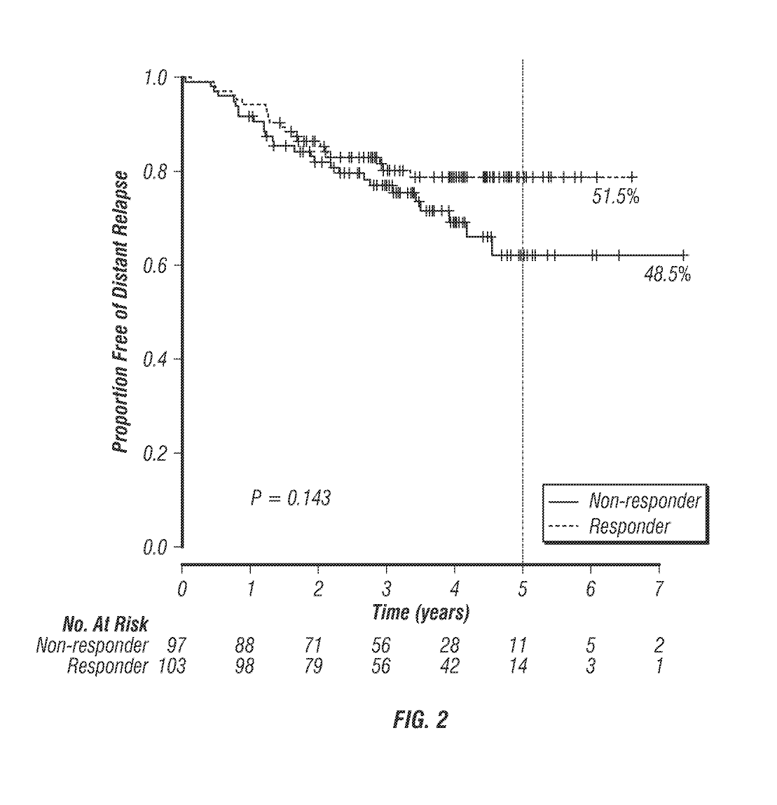

FIG. 2 shows the survival outcomes of patients from the independent validation cohort (Table 1A) that were predicted as good responders by the ER-stratified predictor of response (RCB0/I) described in Example 4. Survival is defined by distant relapse-free survival (DRFS) over a period of about 80 months after the initial diagnostic biopsy. These patients have undergone surgery where it was considered appropriate and the ER-positive patients received hormonal therapy (tamoxifen) for 5 years after the surgery. ER-negative patients did not receive any treatment post-surgery.

The plot shows that predicted responders to taxane-containing chemotherapy (FIG. 2) show fewer events resulting in lower distant relapse rate (˜20% relapse rate after 60 months) whereas the remainder show considerably higher relapse rate among the patients (˜40% relapse rate in after 60 months). The overall separation of the two curves, poor responders corresponding to lower survival and good responders corresponding to higher survival, however, are not statistically significant (log-rank test p=0.143). This indicates that the response-based predictor facilitates some separation according to outcomes after therapy but is not strongly predictive enough on its own to distinctly differentiate survival after therapy in this particular validation cohort.

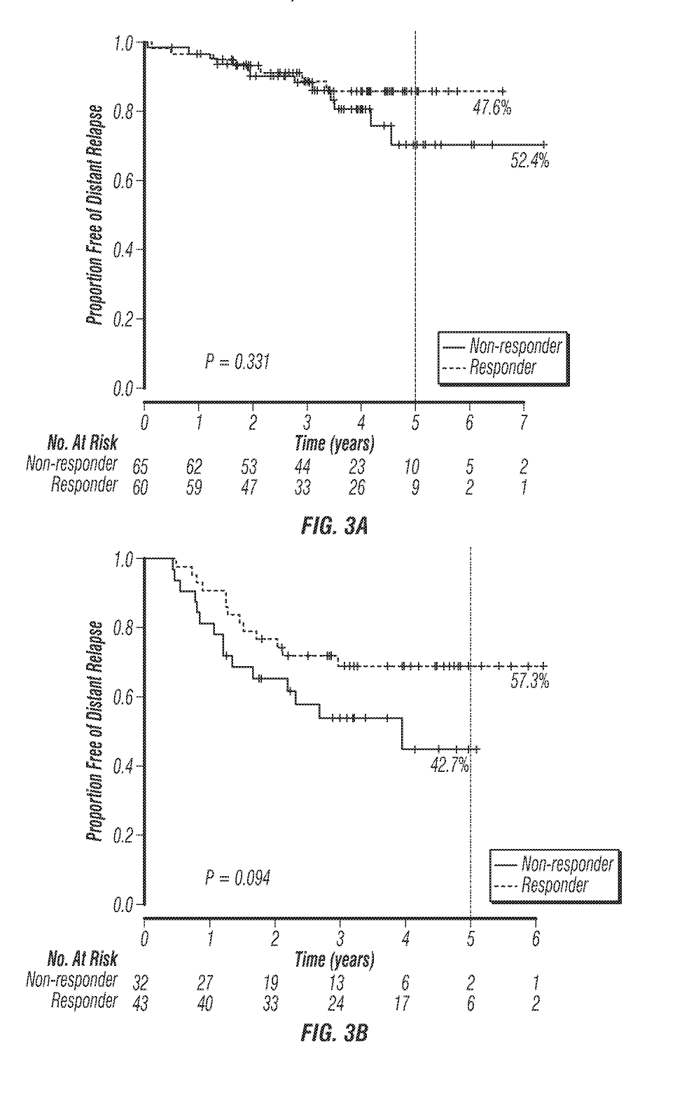

FIG. 3 shows plots of the prediction of the response predictor versus relapse-free survival in ER-positive and ER-negative subsets of the independent validation cohort of Table 1A. The plot shows that predicted responders in ER-positive tumors are not well separated from non-responders over the first 3 years (FIG. 3A), although the predicted non-responders accumulate more events after 3 years, whereas there is a reasonably good separation between responders to taxane-therapy versus non-responders in ER-negative tumors (p=0.094, FIG. 3B). The response-based predictor, therefore, shows a potentially stronger predictive power in ER-negative tumors for outcomes after chemotherapy.

### Example 6

### Prediction of Chemotherapy Outcome Using a Combination of Relapse-Based and Response-Based Predictors

Based on the performance of the relapse-based or resistance predictor of Example 2 and the response-based predictor of Example 4, combined prediction using the two predictors was studied in the validation cohort (Table 1A). The relapse-based predictor was applied first to the cohort as described in FIG. 1 to obtain low-risk and high-risk patients. The response-based predictor was then applied to the low-risk patients to further stratify them into two groups—called High responders and Intermediate responders. The patients previously identified as high-risk by the relapse-based predictor were labeled here as Low responders.

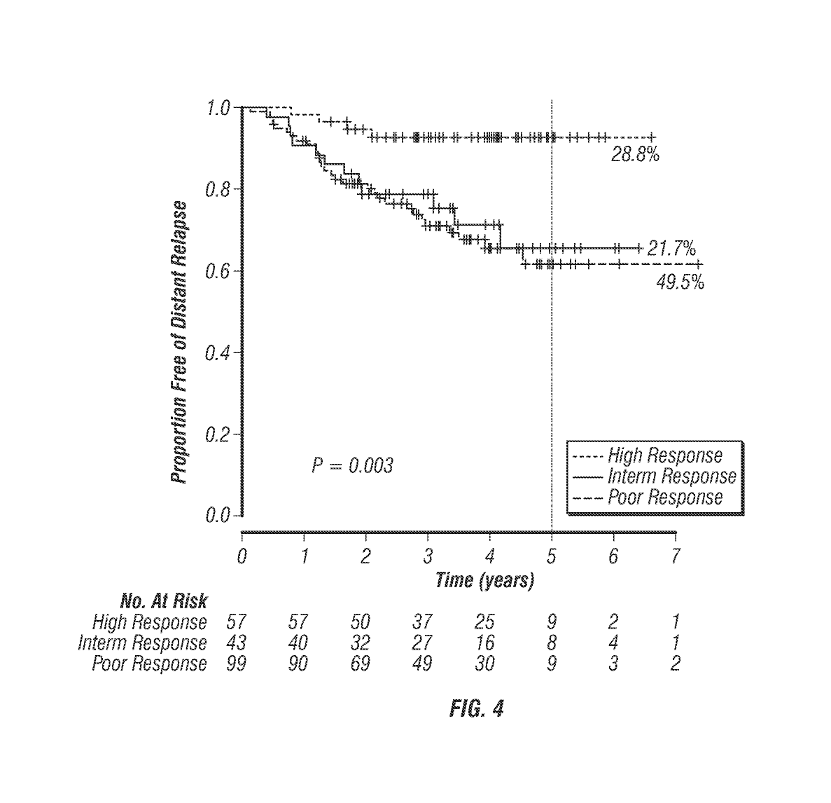

FIG. 4 shows K-M plots of the cohorts defined by the combined predictor based on relapse (resistance) and response. The plot shows about 29% of patients with an excellent 5-year survival (average 92% DRFS at 60 months) versus the Intermediate and Low responders who show approximately 65% or lower DRFS at 60 months. The separation of the curves is statistically significant (p=0.003). The Intermediate and Low responders may be combined into a single group as non-responders since they had very similar DRFS profiles.

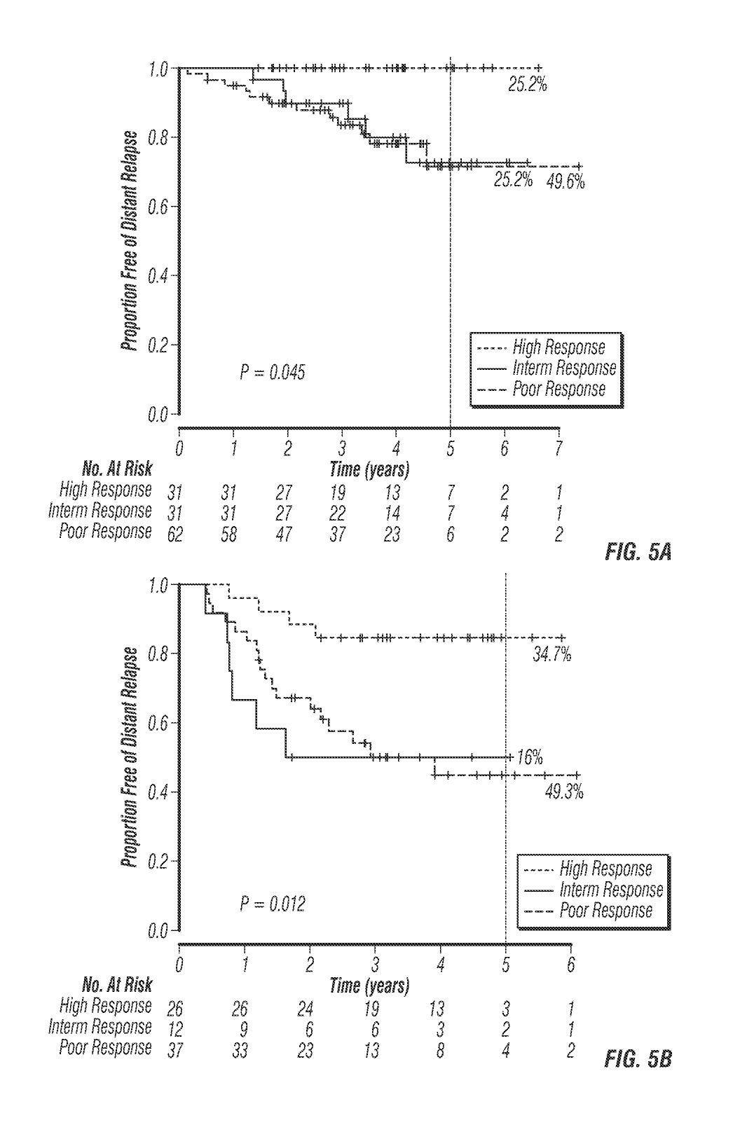

FIG. 5 shows plots of the prediction of the combined predictor versus relapse-free survival in ER-positive (FIG. 5A) and ER-negative (FIG. 5B) subsets of the validation cohort. In both subsets, the High responders as one group are distinctly separated from the Intermediate and Low responders, which together can be considered as Non-responders in both subsets. The responders for the ER-positive tumors have excellent survival (˜100% DRFS at 60 months) versus the non-responders have about 73% DRFS in that time period. The ER-negative tumors, known to have poorer prognosis relative to ER-positive tumors, have an 85% DRFS at 60 months among responders but a much lower DRFS of ˜50% among non-responders. Identifying patients who would be at such high risk despite aggressive chemotherapy would be clinically useful since they can be considered for more advanced therapies or in clinical trials of new therapeutic agents.

### Example 7

### Chemotherapy Outcomes Prediction Using an Index of Endocrine Sensitivity

The prediction of breast cancer sensitivity to endocrine therapy such as tamoxifen and aromatase inhibitors has been described earlier by measurement of gene expression levels (U.S. Provisional Patent Application, 61/174,706). We examined the combination of the sensitivity to endocrine therapy (SET) index with prediction of chemosensitivity using the combined predictor genes described in Example 6.

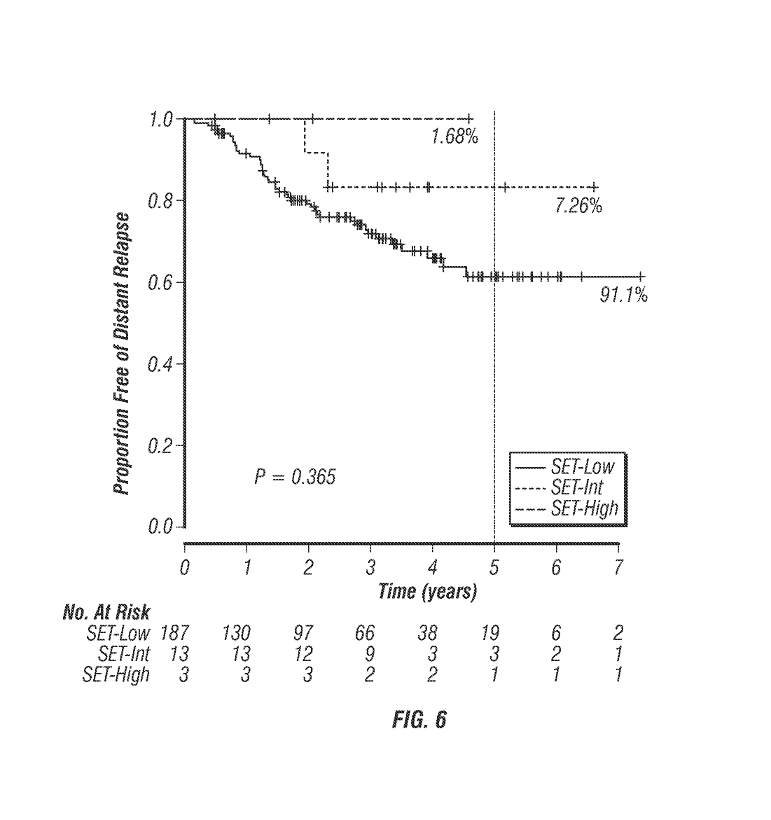

In this example, the endocrine sensitivity index (as described in U.S. 61/174,706) was applied first to the validation cohort of patients shown in Table 1A. The High and Intermediate classes (8.9%) of endocrine sensitivity showed good relapse-free survival (FIG. 6). Therefore, patients who show high and intermediate values of the endocrine sensitivity index will have a good outcome when chemotherapy is combined with endocrine therapy for these patients. The remaining patients (91.1%) need to be evaluated additionally for benefit of chemotherapy using other methods, such as the predictors described in Examples 2 and 4.

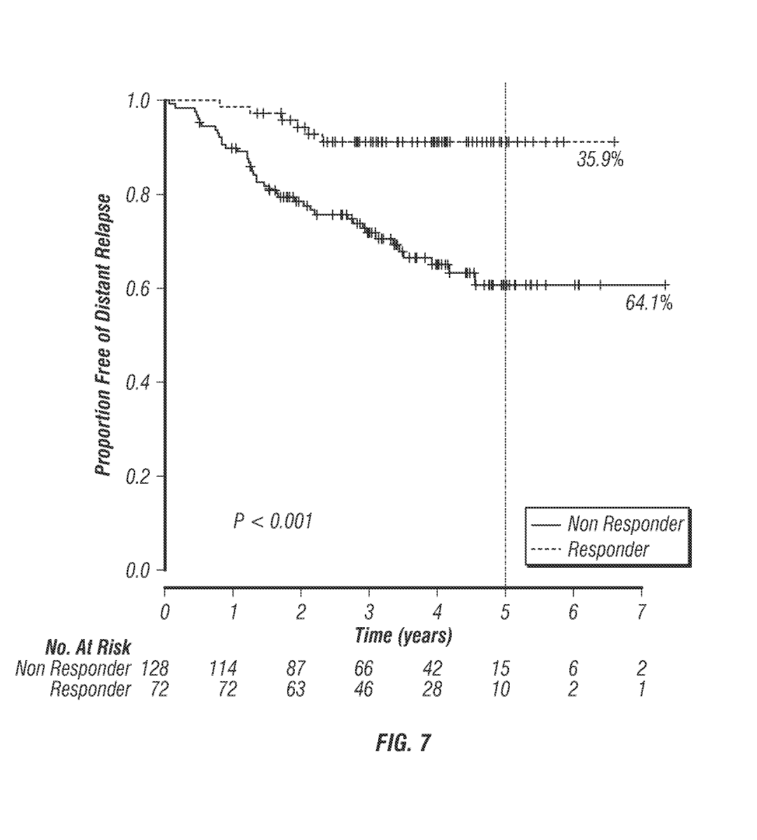

The relapse-based predictor (Example 2) and response-based predictor (Example 4), combined as described in Example 6, were applied to the patient samples classified with a low endocrine sensitivity index. Patients identified for chemosensitivity by the predictors of Example 2 and 4 together were then combined with patients with high and intermediate endocrine sensitivity index as responders. FIG. 7 shows the predicted good and poor responders identified by these combined predictors. The poor responders (64.1% of patients) show a larger number of events resulting in lower DRFS (˜60% relapse-free after 60 months) whereas the responder patients (35.9% of total) show considerably higher relapse-free survival among the patients (˜95% relapse-free after 60 months). The two curves, poor responders corresponding to lower survival and good responders corresponding to higher survival, are statistically distinct (p<0.001). This shows that the synergistic use of genomic indices such as the SET index along with the predictor genes in Tables 2 and 3 can very effectively identify patients who will have a good outcome or a poor outcome as a result of chemotherapy.

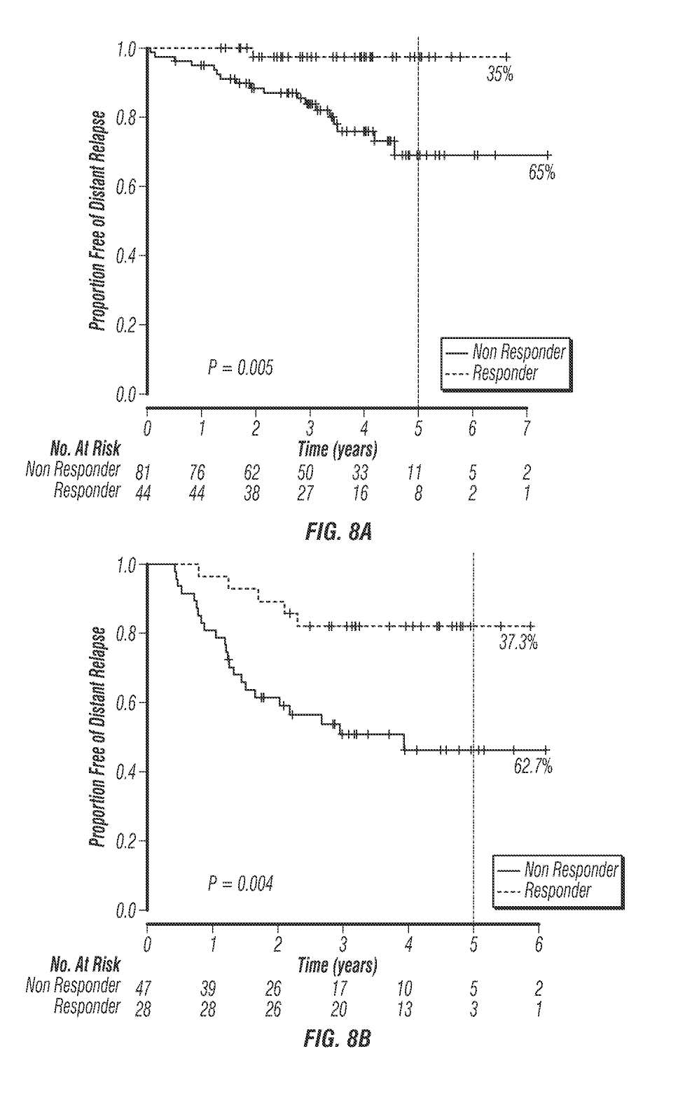

FIG. 8 shows the performance of the combined predictor separately ER positive and ER negative patients. In ER-positive patients (FIG. 8A), the predicted responders have an excellent outcome as ˜98%% relapse-free survival over 5 years and represent about 35% of the patients whereas the poor responders have a relapse-free survival of 65% in comparison. In ER-negative patients (FIG. 8B), the identified responders have about an 80% relapse-free survival rate in contrast to poor responders who do much worse at 45% relapse-free survival. In both sets of patients, whether ER-positive or ER-negative, the responder and non-responder curves are distinctly separated with statistical significance (p=0.005 for ER-positive and p=0.004 for ER− negative subsets, respectively).

### Example 8

### Predictor of Poor Response to Chemotherapy

Patients and Samples—

Patient samples used were those shown in Table 1A. All other laboratory analytic methods were the same as in Example 1.

Methods for Building Predictors of Poor Response to Chemotherapy—

The inventors used the response endpoint RCB-III, representing extensive residual disease after the completion of neoadjuvant chemotherapy, to identify genes that differentiated patients who failed to respond to chemotherapy versus all others in the discovery cohort (Table 1A). Prior to analysis, probes that either had low specificity (those that include extensions_xfri_{—} in their name) or housekeeping probes (those starting with AFFX) were selected and removed from the candidate probesets. This process removed 2522 probesets. Subsequently, a non-specific filter was applied to retain probesets that has log 2-transformed intensity of at least 5 in at least 75% of the arrays. A total of 16289 probesets (73% of all) were retained for further analysis.

The samples in the development cohort were subdivided in ER+ and ER− subsets and in lymph node negative (N0) and lymph positive (NP) subsets within each ER group. Means and standard deviations (SDs) of the 16289 genes were computed for each of the 4 subsets of cases. Within each ER cohort, the means and SDs for N0 and NP subsets were averaged to yield nodal-status adjusted statistics. These means and SDs were then used to scale the expression values of all probesets using the corresponding statistics for ER+ or ER− cases.

Each probeset was evaluated for differential expression in the two responder groups (RCB-III vs rest) using an unequal variance t-statistic based on the trimmed means and trimmed standard deviations in the two groups using a trim fraction of 0.025 (i.e. the lowest 2.5% and highest 2.5% values were eliminated and the statistics were calculated on the remaining 95% of the observations in each group). Degrees of freedom for the unequal variance t-statistic were estimated based on Satterthwaite's approximation (Armitage, Berry & Matthews, 2002). The significance of association of each probe set with response was assessed based on the unequal variance t-statistic. P-values for the significance of each probeset were calculated from the t-distribution with the corresponding degrees of freedom.

To account for sampling variability in the training dataset, the differential expression analysis for each probeset described in the previous paragraph was performed repeatedly using a bootstrap procedure in which cases were sampled with replacement to generate bootstrapped datasets of the same size as the original dataset. This process was repeated 499 times, thus generating 500 estimates for the p-values of each probeset. The association of each probeset with distant relapse risk was assessed within each bootstrapped dataset at a critical significance level of 0.00075 to account for multiple testing. Those probesets that were called significant in at least 30% of the bootstrap replicates were selected as candidate probesets. This process was applied separately to the ER-positive and ER-negative cases in the training dataset and resulted in 256 and 202 candidate probesets in the ER+ and ER-subsets.

In developing the RCB-based chemothereapy response predictor, the inventors used an approach that combines feature selection and model discovery using a multivariate penalized approach called Gradient Directed Regularization developed by Prof. J. Friedman at Stanford University, a description of which can be found on the World Wide Web at stat.stanford.edu/˜jhf/ftp/pathlite.pdf. The informative genes are selected through penalization using the maximization of the area under the ROC curve (AUC) as the optimization criterion. Ma and Huang have previously used a similar approach for disease classification (Ma, 2006).

For predictor discovery and evaluation the inventors followed a cross-validation protocol. First, the input dataset is randomly partitioned into a training set and a test set. A 5-fold cross-validation for a 4:1 split stratified by response group between training and test sets was used (Dudoit, 2002). The training set consisting of ⅘ of the original data is used to develop the predictor. The algorithm starts with the same initial list of candidate genes that were determined through the bootstrap procedure and iteratively refines the predictor by selecting genes that contribute in maximizing the AUC of the candidate predictor. The maximum level of penalization is used to derive the most parsimonious predictors. Since different optimal reporter gene sets might result from the different internal cross-validation folds, the number of times each gene is selected is tracked to provide a measure of its importance or its reliability. The trained predictor is then tested on the ⅕ hold-out part of the training dataset and its performance is evaluated based on the AUC.

The entire process of randomly splitting the data to a training- and a test-set was repeated 499 times to obtain the distributions and summary statistics of the performance metrics from the cross-validated replicates.

The final predictors for ER+ and ER− subsets used 73 probesets and 54 probesets respectively to make the predictions. The probesets, genes that they encode for, and their weights (coefficients) are shown in Table 4. The risk score is calculated by multiplying the scaled log 2-transformed expression level of each gene in a given sample by its corresponding weight and then adding up the weighted expression values for all genes in the signature. The following formula describes the score calculation for sample i:

$${y}_{i} = \{ \begin{matrix}{\sum }_{j = 1}^{{K}^{+}} {w}_{j}^{+} {z}_{ij}^{+} , & if ER positive \\ {\sum }_{j = 1}^{{K}^{-}} {w}_{j}^{-} {z}_{ij}^{-} , & if ER negative\end{matrix}$$

where wⱼ is the weight of gene j in the signature, zᵢⱼ is the log 2-transformed and scaled expression value of gene j in sample i, K is the number of genes in the signature, and the + or − symbols refer to the ER+ and ER− signatures.

A cut point was selected to dichotomize the risk score and predict two risk classes. The optimal cutoff was selected in order to maximize the accuracy of the prediction. A cutoff of 0 was selected for both the ER+ and ER− scores. Positive scores signify “resistant” or poor-responder and a zero or negative score signifies “non-resistant”.

TABLE 4
Genes used for prediction of poor response, RCB-III, in ER-stratified patient
subsets

| | | | | | | |
|---|---|---|---|---|---|---|
| Probe Set | Symbol | Description | GeneID | Chromosome | Cytoband | Weight |

ER-Positive

| | | | | | | | |
|---|---|---|---|---|---|---|---|
| 1 | 200045_at | ABCF1 | ATP-binding cassette, | 23 | 6 | 6p21.33 | −0.1287 |
| | | | sub-family F (GCN20), | | | | |
| | | | member 1 | | | | |
| 2 | 218868_at | ACTR3B | ARP3 actin-related | 57180 | 7 | 7q36.1 | −0.073 |
| | | | protein 3 homolog B | | | | |
| | | | (yeast) | | | | |
| 3 | 213532_at | ADAM17 | ADAM | 6868 | 2 | 2p25 | −0.3194 |
| | | | metallopeptidase | | | | |
| | | | domain 17 | | | | |
| 4 | 217090_at | ADAM3A | ADAM | 1587 | 8 | 8p11.23 | 0.3763 |
| | | | metallopeptidase | | | | |
| | | | domain 3A (cyritestin | | | | |
| | | | 1) | | | | |
| 5 | 205013_s_at | ADORA2A | adenosine A2a | 135 | 22 | 22q11.23 | 0.1786 |
| | | | receptor | | | | |
| 6 | 208042_at | AGGF1 | angiogenic factor with | 55109 | 5 | 5q13.3 | 0.1425 |
| | | | G patch and FHA | | | | |
| | | | domains 1 | | | | |
| 7 | 215789_s_at | AJAP1 | adherens junctions | 55966 | 1 | 1p36.32 | −0.111 |
| | | | associated protein 1 | | | | |
| 8 | 221825_at | ANGEL2 | angel homolog 2 | 90806 | 1 | 1q32.3 | 0.5463 |
| | | | (Drosophila) | | | | |
| 9 | 202631_s_at | APPBP2 | amyloid beta | 10513 | 17 | 17q21-q23 | −0.1027 |
| | | | precursor protein | | | | |
| | | | (cytoplasmic tail) | | | | |
| | | | binding protein 2 | | | | |
| 10 | 200011_s_at | ARF3 | ADP-ribosylation | 377 | 12 | 12q13 | 0.3083 |
| | | | factor 3 | | | | |
| 11 | 202492_at | ATG9A | ATG9 autophagy | 79065 | 2 | 2q35 | −0.6807 |
| | | | related 9 homolog A | | | | |
| | | | (S. cerevisiae) | | | | |
| 12 | 212930_at | ATP2B1 | ATPase, Ca++ | 490 | 12 | 12q21.3 | 0.0737 |
| | | | transporting, plasma | | | | |
| | | | membrane 1 | | | | |
| 13 | 218789_s_at | C11orf71 | chromosome 11 open | 54494 | 11 | 11q14.2-q14.3 | 0.2824 |
| | | | reading frame 71 | | | | |
| 14 | 219022_at | C12orf43 | chromosome 12 open | 64897 | 12 | 12q | 0.3528 |
| | | | reading frame 43 | | | | |
| 15 | 214322_at | CAMK2G | calcium/calmodulin- | 818 | 10 | 10q22 | −0.0176 |
| | | | dependent protein | | | | |
| | | | kinase II gamma | | | | |
| 16 | 218384_at | CARHSP1 | calcium regulated | 23589 | 16 | 16p13.2 | 0.5253 |
| | | | heat stable protein 1, | | | | |
| | | | 24 kDa | | | | |
| 17 | 212586_at | CAST | calpastatin | 831 | 5 | 5q15 | −0.0498 |
| 18 | 218592_s_at | CECR5 | cat eye syndrome | 27440 | 22 | | −0.4437 |
| | | | chromosome region, | | | | |
| | | | candidate 5 | | | | |
| 19 | 218439_s_at | COMMD10 | COMM domain | 51397 | 5 | 5q23.1 | 0.1117 |
| | | | containing 10 | | | | |
| 20 | 211808_s_at | CREBBP | CREB binding protein | 1387 | 16 | 16p13.3 | 0.1494 |
| 21 | 209164_s_at | CYB561 | cytochrome b-561 | 1534 | 17 | 17q11-qter | −0.3429 |
| 22 | 203979_at | CYP27A1 | cytochrome P450, | 1593 | 2 | 2q33-qter | −0.3785 |
| | | | family 27, subfamily A, | | | | |
| | | | polypeptide 1 | | | | |
| 23 | 216874_at | DKFZp686O1327 | hypothetical gene | 401014 | 2 | 2q22.3 | 1 |
| | | | supported by | | | | |
| | | | BC043549; BX648102 | | | | |
| 24 | 204797_s_at | EML1 | echinoderm | 2009 | 14 | 14q32 | 0.2037 |
| | | | microtubule | | | | |
| | | | associated protein like 1 | | | | |
| 25 | 218692_at | GOLSYN | Golgi-localized protein | 55638 | 8 | 8q23.2 | 0.174 |
| 26 | 202453_s_at | GTF2H1 | general transcription | 2965 | 11 | 11p15.1-p14 | −0.3144 |
| | | | factor IIH, polypeptide | | | | |
| | | | 1, 62 kDa | | | | |
| 27 | 221046_s_at | GTPBP8 | GTP-binding protein 8 | 29083 | 3 | 3q13.2 | −0.118 |
| | | | (putative) | | | | |
| 28 | 208886_at | H1F0 | H1 histone family, | 3005 | 22 | 22q13.1 | 0.028 |
| | | | member 0 | | | | |
| 29 | 205426_s_at | HIP1 | huntingtin interacting | 3092 | 7 | 7q11.23 | 0.4815 |
| | | | protein 1 | | | | |
| 30 | 202983_at | HLTF | helicase-like | 6596 | 3 | 3q25.1-q26.1 | −0.1866 |
| | | | transcription factor | | | | |
| 31 | 217145_at | IGKC | immunoglobulin kappa | 3514 | 2 | 2p12 | −0.035 |
| | | | constant | | | | |
| 32 | 204863_s_at | IL6ST | interleukin 6 signal | 3572 | 5 | 5q11 | −0.6475 |
| | | | transducer (gp130, | | | | |
| | | | oncostatin M receptor) | | | | |
| 33 | 211817_s_at | KCNJ5 | potassium inwardly- | 3762 | 11 | 11q24 | 0.3023 |
| | | | rectifying channel, | | | | |
| | | | subfamily J, member 5 | | | | |
| 34 | 201776_s_at | KIAA0494 | KIAA0494 | 9813 | 1 | 1pter-p22.1 | −0.3831 |
| 35 | 209212_s_at | KLF5 | Kruppel-like factor 5 | 688 | 13 | 13q22.1 | −0.1623 |
| | | | (intestinal) | | | | |
| 36 | 212271_at | MAPK1 | mitogen-activated | 5594 | 22 | 22q11.2 | 0.1979 |
| | | | protein kinase 1 | | | | |
| 37 | 206904_at | MATN1 | matrilin 1, cartilage | 4146 | 1 | 1p35 | −0.4397 |
| | | | matrix protein | | | | |
| 38 | 206961_s_at | MED20 | mediator complex | 9477 | 6 | 6p21.1 | 0.1547 |
| | | | subunit 20 | | | | |
| 39 | 213403_at | MFSD9 | major facilitator | 84804 | 2 | 2q12.1 | 0.3304 |
| | | | superfamily domain | | | | |
| | | | containing 9 | | | | |
| 40 | 209733_at | MID2 | midline 2 | 11043 | X | Xq22.3 | 0.1227 |
| 41 | 218205_s_at | MKNK2 | MAP kinase | 2872 | 19 | 19p13.3 | 0.1801 |
| | | | interacting | | | | |
| | | | serine/threonine | | | | |
| | | | kinase 2 | | | | |
| 42 | 209973_at | NFKBIL1 | nuclear factor of | 4795 | 6 | 6p21.3 | −0.0068 |
| | | | kappa light | | | | |
| | | | polypeptide gene | | | | |
| | | | enhancer in B-cells | | | | |
| | | | inhibitor-like 1 | | | | |
| 43 | 217963_s_at | NGFRAP1 | nerve growth factor | 27018 | X | Xq22.2 | 0.201 |
| | | | receptor (TNFRSF16) | | | | |
| | | | associated protein 1 | | | | |
| 44 | 207400_at | NPY5R | neuropeptide Y | 4889 | 4 | 4q31-q32 | 0.0984 |
| | | | receptor Y5 | | | | |
| 45 | 202097_at | NUP153 | nucleoporin 153 kDa | 9972 | 6 | 6p22.3 | −0.1197 |
| 46 | 220631_at | OSGEPL1 | O-sialoglycoprotein | 64172 | 2 | 2q32.2 | 0.2148 |
| | | | endopeptidase-like 1 | | | | |
| 47 | 205077_s_at | PIGF | phosphatidylinositol | 5281 | 2 | 2p21-p16 | 0.5495 |
| | | | glycan anchor | | | | |
| | | | biosynthesis, class F | | | | |
| 48 | 220811_at | PRG3 | proteoglycan 3 | 10394 | 11 | 11q12 | 0.2689 |
| 49 | 208733_at | RAB2A | RAB2A, member RAS | 5862 | 8 | 8q12.1 | 0.581 |
| | | | oncogene family | | | | |
| 50 | 206066_s_at | RAD51C | RAD51 homolog C (S. cerevisiae) | 5889 | 17 | 17q22-q23 | −0.0517 |
| 51 | 206290_s_at | RGS7 | regulator of G-protein | 6000 | 1 | 1q23.1 | 0.0092 |
| | | | signaling 7 | | | | |
| 52 | 214519_s_at | RLN2 | relaxin 2 | 6019 | 9 | 9p24.1 | 0.103 |
| 53 | 206805_at | SEMA3A | sema domain, | 10371 | 7 | 7p12.1 | 0.1132 |
| | | | immunoglobulin | | | | |
| | | | domain (Ig), short | | | | |
| | | | basic domain, | | | | |
| | | | secreted, | | | | |
| | | | (semaphorin) 3A | | | | |
| 54 | 208941_s_at | SEPHS1 | selenophosphate | 22929 | 10 | 10p14 | −0.6301 |
| | | | synthetase 1 | | | | |
| 55 | 213755_s_at | SKI | v-ski sarcoma viral | 6497 | 1 | 1q22-q24 | −0.1078 |
| | | | oncogene homolog | | | | |
| | | | (avian) | | | | |
| 56 | 202667_s_at | SLC39A7 | solute carrier family | 7922 | 6 | 6p21.3 | −0.1376 |
| | | | 39 (zinc transporter), | | | | |
| | | | member 7 | | | | |
| 57 | 216611_s_at | SLC6A2 | solute carrier family 6 | 6530 | 16 | 16q12.2 | −0.0064 |
| | | | (neurotransmitter | | | | |
| | | | transporter, | | | | |
| | | | noradrenalin), | | | | |
| | | | member 2 | | | | |
| 58 | 211805_s_at | SLC8A1 | solute carrier family 8 | 6546 | 2 | 2p23-p22 | −0.248 |
| | | | (sodium/calcium | | | | |
| | | | exchanger), member 1 | | | | |
| 59 | 205596_s_at | SMURF2 | SMAD specific E3 | 64750 | 17 | 17q22-q23 | −0.1446 |
| | | | ubiquitin protein ligase 2 | | | | |
| 60 | 203054_s_at | TCTA | T-cell leukemia | 6988 | 3 | 3p21 | 0.2818 |
| | | | translocation altered | | | | |
| | | | gene | | | | |
| 61 | 218099_at | TEX2 | testis expressed 2 | 55852 | 17 | 17q23.3 | −0.0149 |
| 62 | 217121_at | TNKS | tankyrase, TRF1- | 8658 | 8 | 8p23.1 | −0.5943 |
| | | | interacting ankyrin- | | | | |
| | | | related ADP-ribose | | | | |
| | | | polymerase | | | | |
| 63 | 220415_at | TNNI3K | TNNI3 interacting | 51086 | 1 | 1p31.1 | 0.3122 |
| | | | kinase | | | | |
| 64 | 209593_s_at | TOR1B | torsin family 1, | 27348 | 9 | 9q34 | −0.0834 |
| | | | member B (torsin B) | | | | |
| 65 | 215796_at | TRD@ | T cell receptor delta | 6964 | 14 | 14q11.2 | 0.4491 |
| | | | locus | | | | |
| 66 | 210541_s_at | TRIM27 | tripartite motif- | 5987 | 6 | 6p22 | −0.0174 |
| | | | containing 27 | | | | |
| 67 | 213563_s_at | TUBGCP2 | tubulin, gamma | 10844 | 10 | 10q26.3 | −0.169 |
| | | | complex associated | | | | |
| | | | protein 2 | | | | |
| 68 | 221839_s_at | UBAP2 | ubiquitin associated | 55833 | 9 | 9p13.3 | −0.0133 |
| | | | protein 2 | | | | |
| 69 | 213822_s_at | UBE3B | ubiquitin protein ligase | 89910 | 12 | 12q24.11 | −0.4683 |
| | | | E3B | | | | |
| 70 | 221746_at | UBL4A | ubiquitin-like 4A | 8266 | X | Xq28 | −0.0227 |
| 71 | 219740_at | VASH2 | vasohibin 2 | 79805 | 1 | 1q32.3 | −0.1995 |
| 72 | 205877_s_at | ZC3H7B | zinc finger CCCH-type | 23264 | 22 | 22q13.2 | −0.9818 |
| | | | containing 7B | | | | |
| 73 | 218413_s_at | ZNF639 | zinc finger protein 639 | 51193 | 3 | 3q26.33 | −0.1572 |

ER-Negative

| | | | | | | | |
|---|---|---|---|---|---|---|---|
| 1 | 214919_s_at | ANKHD1- | ANKHD1- | 404734 | 5 | 5q31.3 | 0.1134 |
| | | EIF4EBP3 | EIF4EBP3 | | | | |
| | | | readthrough | | | | |
| 2 | 202955_s_at | ARFGEF1 | ADP-ribosylation | 10565 | 8 | 8q13 | 0.0616 |
| | | | factor guanine | | | | |
| | | | nucleotide | | | | |
| | | | exchange factor | | | | |
| | | | 1(brefeldin A- | | | | |
| | | | inhibited) | | | | |
| 3 | 203576_at | BCAT2 | branched chain | 587 | 19 | 19q13 | −0.1544 |
| | | | aminotransferase | | | | |
| | | | 2, mitochondrial | | | | |
| 4 | 202047_s_at | CBX6 | chromobox | 23466 | 22 | 22q13.1 | 0.0673 |
| | | | homolog 6 | | | | |
| 5 | 220674_at | CD22 | CD22 molecule | 933 | 19 | 19q13.1 | 0.1582 |
| 6 | 208022_s_at | CDC14B | CDC14 cell | 8555 | 9 | 9q22.32-q22.33 | −0.4312 |
| | | | division cycle 14 | | | | |
| | | | homolog B (S. cerevisiae) | | | | |
| 7 | 204250_s_at | CEP164 | centrosomal | 22897 | 11 | 11q23.3 | 0.1607 |
| | | | protein 164 kDa | | | | |
| 8 | 218597_s_at | CISD1 | CDGSH iron sulfur | 55847 | 10 | 10q21.1 | 0.6177 |
| | | | domain 1 | | | | |
| 9 | 206073_at | COLQ | collagen-like tail | 8292 | 3 | 3p25 | −0.033 |
| | | | subunit (single | | | | |
| | | | strand of | | | | |
| | | | homotrimer) of | | | | |
| | | | asymmetric | | | | |
| | | | acetylcholinesterase | | | | |
| 10 | 208303_s_at | CRLF2 | cytokine receptor | 64109 | X, Y | Xp22.3 | −0.0413 |
| | | | like factor 2 | | | | |
| 11 | 217047_s_at | FAM13A | family with | 10144 | 4 | 4q22.1 | −0.1603 |
| | | | sequence | | | | |
| | | | similarity 13, | | | | |
| | | | member A | | | | |
| 12 | 212484_at | FAM89B | family with | 23625 | 11 | 11q23 | −0.0232 |
| | | | sequence | | | | |
| | | | similarity 89, | | | | |
| | | | member B | | | | |
| 13 | 204437_s_at | FOLR1 | folate receptor 1 | 2348 | 11 | 11q13.3-q14.1 | −1 |
| | | | (adult) | | | | |
| 14 | 203314_at | GTPBP6 | GTP binding | 8225 | X, Y | Xp22.33 | 0.0926 |
| | | | protein 6 (putative) | | | | |
| 15 | 210964_s_at | GYG2 | glycogenin 2 | 8908 | X | Xp22.3 | −0.364 |
| 16 | 212431_at | HMGXB3 | HMG box domain | 22993 | 5 | 5q32 | 0.0875 |
| | | | containing 3 | | | | |
| 17 | 211616_s_at | HTR2A | 5- | 3356 | 13 | 13q14-q21 | 0.1049 |
| | | | hydroxytryptamine | | | | |
| | | | (serotonin) | | | | |
| | | | receptor 2A | | | | |
| 18 | 204990_s_at | ITGB4 | integrin, beta 4 | 3691 | 17 | 17q25 | −0.2769 |
| 19 | 207012_at | MMP16 | matrix | 4325 | 8 | 8q21.3 | 0.1034 |
| | | | metallopeptidase | | | | |
| | | | 16 (membrane- | | | | |
| | | | inserted) | | | | |
| 20 | 212251_at | MTDH | Metadherin | 92140 | 8 | 8q22.1 | 0.7935 |
| 21 | 202039_at | MYO18A | myosin XVIIIA | 399687 | 17 | 17q11.2 | 0.1596 |
| 22 | 222018_at | NACA | nascent | 4666 | 12 | 12q23-q24.1 | 0.1843 |
| | | | polypeptide- | | | | |
| | | | associated | | | | |
| | | | complex alpha | | | | |
| | | | subunit | | | | |
| 23 | 209519_at | NCBP1 | nuclear cap | 4686 | 9 | 9q34.1 | −0.4186 |
| | | | binding protein | | | | |
| | | | subunit 1, 80 kDa | | | | |
| 24 | 213032_at | NFIB | nuclear factor I/B | 4781 | 9 | 9p24.1 | 0.1829 |
| 25 | 215818_at | NUDT7 | nudix (nucleoside | 283927 | 16 | 16q23.1 | −0.1766 |
| | | | diphosphate linked | | | | |
| | | | moiety X)-type | | | | |
| | | | motif 7 | | | | |
| 26 | 218271_s_at | PARL | presenilin | 55486 | 3 | 3q27.1 | −0.0708 |
| | | | associated, | | | | |
| | | | rhomboid-like | | | | |
| 27 | 204049_s_at | PHACTR2 | phosphatase and | 9749 | 6 | 6q24.2 | 0.1352 |
| | | | actin regulator 2 | | | | |
| 28 | 217806_s_at | POLDIP2 | polymerase (DNA- | 26073 | 17 | 17q11.2 | 0.3128 |
| | | | directed), delta | | | | |
| | | | interacting protein 2 | | | | |
| 29 | 206653_at | POLR3G | polymerase (RNA) | 10622 | 5 | 5q14.3 | −0.3632 |
| | | | III (DNA directed) | | | | |
| | | | polypeptide G | | | | |
| | | | (32 kD) | | | | |
| 30 | 210831_s_at | PTGER3 | prostaglandin E | 5733 | 1 | 1p31.2 | −0.0066 |
| | | | receptor 3 | | | | |
| | | | (subtype EP3) | | | | |
| 31 | 213933_at | PTGER3 | prostaglandin E | 5733 | 1 | 1p31.2 | 0.0187 |
| | | | receptor 3 | | | | |
| | | | (subtype EP3) | | | | |
| 32 | 208393_s_at | RAD50 | RAD50 homolog | 10111 | 5 | 5q31 | −0.1057 |
| | | | (S. cerevisiae) | | | | |
| 33 | 221705_s_at | SIKE1 | suppressor of | 80143 | 1 | 1p13.2 | −0.2882 |
| | | | IKBKE 1 | | | | |
| 34 | 211112_at | SLC12A4 | solute carrier | 6560 | 16 | 16q22.1 | −0.1596 |
| | | | family 12 | | | | |
| | | | (potassium/chloride | | | | |
| | | | transporters), | | | | |
| | | | member 4 | | | | |
| 35 | 215294_s_at | SMARCA1 | SWI/SNF related, | 6594 | X | Xq25 | 0.056 |
| | | | matrix associated, | | | | |
| | | | actin dependent | | | | |
| | | | regulator of | | | | |
| | | | chromatin, | | | | |
| | | | subfamily a, | | | | |
| | | | member 1 | | | | |
| 36 | 215458_s_at | SMURF1 | SMAD specific E3 | 57154 | 7 | 7q22.1 | −0.1767 |
| | | | ubiquitin protein | | | | |
| | | | ligase 1 | | | | |
| 37 | 215860_at | SYT12 | synaptotagmin XII | 91683 | 11 | 11q13.2 | −0.023 |
| 38 | 222173_s_at | TBC1D2 | TBC1 domain | 55357 | 9 | 9q22.33 | −0.124 |
| | | | family, member 2 | | | | |
| 39 | 204147_s_at | TFDP1 | transcription factor | 7027 | 13 | 13q34 | 0.1725 |
| | | | Dp-1 | | | | |
| 40 | 206260_at | TGM4 | transglutaminase | 7047 | 3 | 3p22-p21.33 | 0.2701 |
| | | | 4 (prostate) | | | | |
| 41 | 212963_at | TM2D1 | TM2 domain | 83941 | 1 | 1p31.3 | 0.1779 |
| | | | containing 1 | | | | |
| 42 | 213882_at | TM2D1 | TM2 domain | 83941 | 1 | 1p31.3 | 0.1487 |
| | | | containing 1 | | | | |
| 43 | 219182_at | TMEM231 | transmembrane | 79583 | 16 | 16q23.1 | −0.2436 |
| | | | protein 231 | | | | |
| 44 | 209344_at | TPM4 | tropomyosin 4 | 7171 | 19 | 19p13.1 | 0.3404 |
| 45 | 217056_at | TRD@ | T cell receptor | 6964 | 14 | 14q11.2 | 0.0697 |
| | | | delta locus | | | | |
| 46 | 217065_at | TRD@ | T cell receptor | 6964 | 14 | 14q11.2 | 0.0128 |
| | | | delta locus | | | | |
| 47 | 203701_s_at | TRMT1 | TRM1 tRNA | 55621 | 19 | 19p13.2 | 0.187 |
| | | | methyltransferase | | | | |
| | | | 1 homolog (S. cerevisiae) | | | | |
| 48 | 201797_s_at | VARS | valyl-tRNA | 7407 | 6 | 6p21.3 | −0.5888 |
| | | | synthetase | | | | |
| 49 | 208453_s_at | XPNPEP1 | X-prolyl | 7511 | 10 | 10q25.3 | 0.5107 |
| | | | aminopeptidase | | | | |
| | | | (aminopeptidase | | | | |
| | | | P) 1, soluble | | | | |
| 50 | 213081_at | ZBTB22 | zinc finger and | 9278 | 6 | 6p21.3 | 0.3968 |
| | | | BTB domain | | | | |
| | | | containing 22 | | | | |
| 51 | 206448_at | ZNF365 | zinc finger protein | 22891 | 10 | 10q21.2 | 0.3809 |
| | | | 365 | | | | |
| 52 | 212867_at | — | — | — | 8 | 8q13.3 | 0.4115 |
| 53 | 213879_at | — | — | — | 17 | 17q25.1 | −0.4574 |
| 54 | 222174_at | — | — | — | 14 | — | 0.017 |

### Example 9

### Prediction of Chemotherapy Outcomes Combining Poor Response as Endpoint

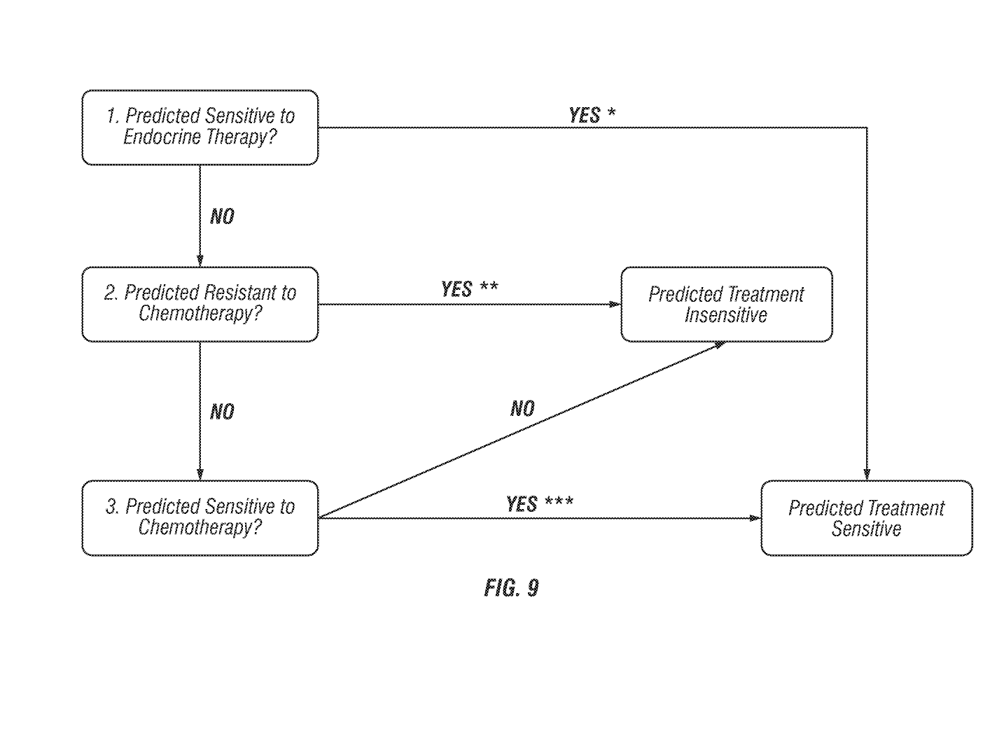

Survival outcomes of patients predicted as responders and non-responders were assessed by using the predictor of RCB-III described in Example 8 used as a combined algorithm with predictors of Examples 2 and 4 and the sensitivity to endocrine therapy (SET) index of Example 7. Survival is defined by distant relapse-free survival (DRFS) over a period of about 80 months. These patients have undergone surgery where it was considered appropriate and the ER-positive patients received hormonal therapy (tamoxifen) for 5 years after the surgery. ER-negative patients did not receive any treatment post-surgery. We combined the individual predictions into a testing algorithm (FIG. 9) for predicted sensitivity to adjuvant treatment of HER2-negative breast cancer with taxane-anthracycline chemotherapy: 1) sensitivity to endocrine therapy (SET) assessed based on the published 165-gene index of the most ER-correlated genes (high or intermediate SET index) that independently predicts survival following adjuvant endocrine or chemoendocrine therapy¹³; 2) resistance to chemotherapy predicted either by early distant relapse events or by extensive residual disease after neoadjuvant chemotherapy; and 3) sensitivity (pathologic response) to chemotherapy.

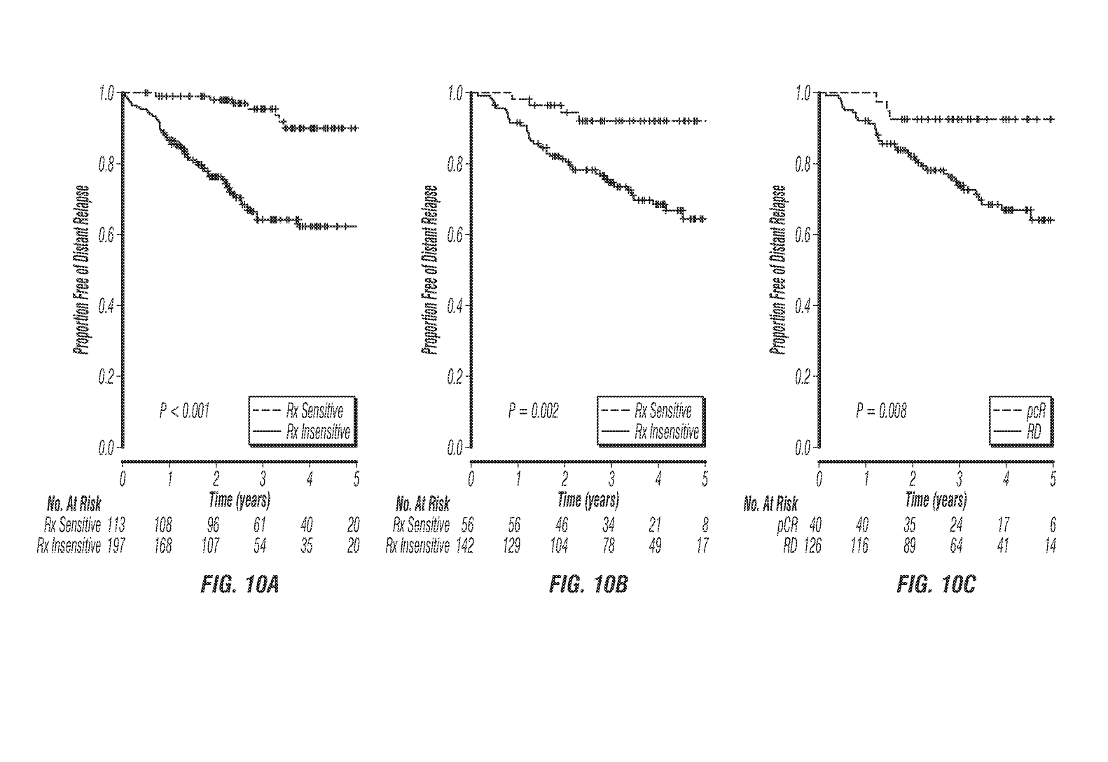

The predictive test (algorithm) was applied to the discovery cohort of 310 samples (FIG. 10A) and then evaluated in the independent validation cohort of 198 patients (99% clinical Stage II-III) who received sequential taxane-anthracycline chemotherapy then endocrine therapy (if ER+). The validation cohort had a pathologic response rate of pCR 25% and of pCR or RCB-I 30%, median follow up of 3 years, and an average 3-year baseline DRFS of 79% (95% CI 74 to 85). The 3-year DRFS (NPV) was 92% (95% CI 85 to 100), and there was significant absolute risk reduction (ARR) of 18% (95% CI 6 to 28), in 28% of patients who were predicted to be treatment-sensitive. The 3-year point estimate of DRFS for those predicted to be treatment-insensitive was 75% (95% CI 67 to 82). Overall, we observed a significant association between predicted sensitivity to treatment and DRFS (p=0.002; FIG. 10B). In 91 tumors with low SET and evaluated for RCB, excellent response from chemotherapy (pCR or RCB-I) was observed in 56% (95% CI 31 to 78) of those predicted to be treatment-sensitive.

Of note, 3-year DRFS in patients predicted to be treatment-sensitive at the time of diagnosis was similar to the 3-year DRFS of 93% (95% CI 85 to 100) in the 21% of patients in the validation cohort who achieved pathologic complete response (pCR) after completion of neoadjuvant chemotherapy. Also, 3-year DRFS for predicted treatment-insensitive was identical to the 3-year DRFS of 75% (95% CI 68 to 83) in those who had residual disease (RD) (FIG. 10C). Furthermore, DRFS estimates for the predicted treatment-sensitive and the actual pCR groups were unchanged at 5 years, and were identical at 65% (95% CI 56 to 75) for the predicted treatment-insensitive and for the actual RD groups.

Treatment Sensitivity According to ER Status:

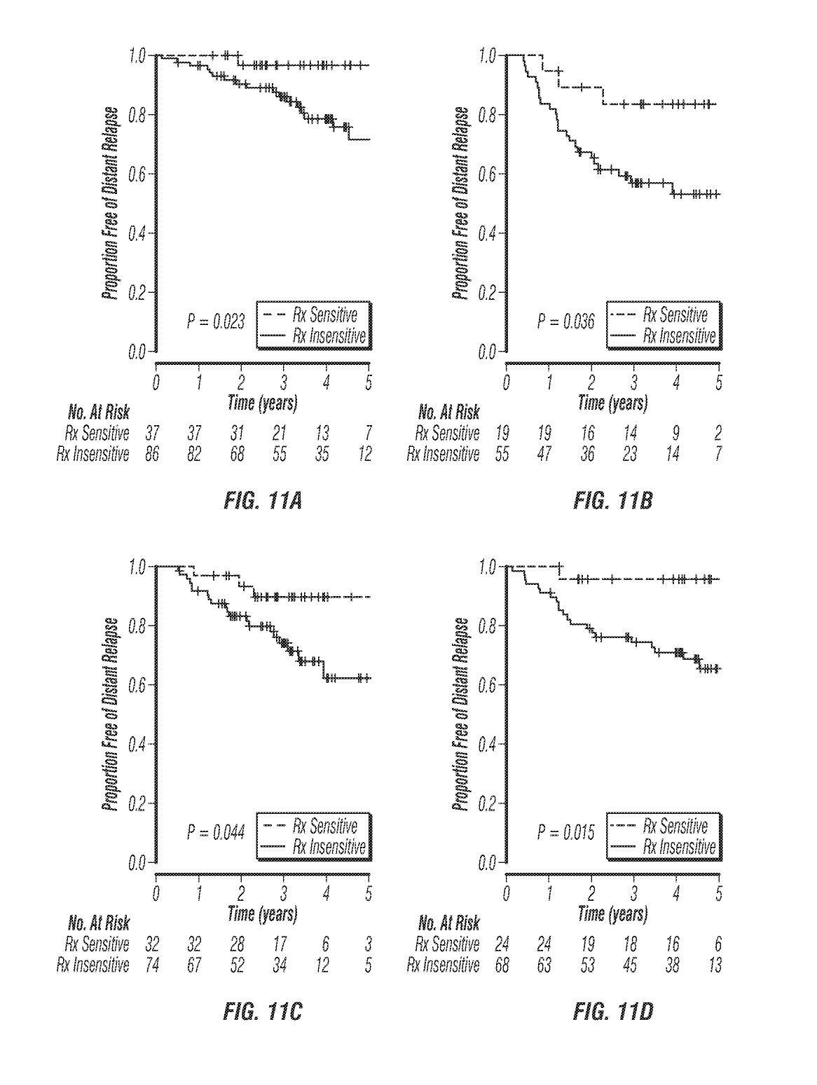

There were 30% and 26% of patients with predicted sensitivity to treatment in the ER+/HER2− and ER−/HER2− subsets, respectively, and both had significantly favorable prognosis (FIG. 11A-B). The treatment sensitive patients identified by test in the ER+/HER2− subset had excellent DRFS (NPV) of 97% (95% CI 91 to 100) and a significant ARR of 11% (95% CI 0.1 to 21) at 3 years of follow up. In the low SET subset of ER+/HER2−, PPV for pathologic response was 42% (95% CI 15 to 72) in 20% who were predicted treatment-sensitive. For ER−/HER2− patients, the PPV for 3-year relapse was 43% (95% CI 28 to 55) if predicted treatment-insensitive. Patients predicted to be treatment-sensitive had considerably improved 3-year DRFS (NPV 83% (95% CI 68 to 100)) and significant ARR of 26% (95% CI 4 to 48) overall, and PPV for pathologic response of 83% (95% CI 36-100).

Performance of the Predictive Test in Other Relevant Subsets

The association between predicted treatment sensitivity and DRFS appears to be unrelated to the type of taxane therapy administered (FIG. 11C-D). The 3-year DRFS was 90% (95% CI 80 to 100) in the subset who received 12 cycles of weekly paclitaxel, and 96% (95% CI 88 to 100) for 4 cycles of 3-weekly docetaxel with capecitabine. Also, the 3-year DRFS was 93% (95% CI 84 to 100) in 128 clinically node-positive patients, with significantly improved DRFS compared to those predicted to be insensitive (p=0.003). The 3-year DRFS was 91% (95% CI 81 to 100) in 70 clinically node-negative patients, but was not significantly different from predicted insensitivity.

Comparison of the Predictive Test with Clinical-Pathologic Parameters

Genomic predictions were independently and significantly associated with risk of distant relapse or death (sensitive versus insensitive; HR 0.19; 95% CI 0.07 to 0.55; p=0.002), after adjusting for standard clinical-pathologic parameters (Table 5). Addition of the genomic prediction to a multivariate Cox model of the clinical-pathologic factors significantly increased the model's predictive utility (likelihood ratio of complete model versus clinical model 13.8, p<0.001). In this model, higher clinical tumor stage (tumor stage T3 or T4 versus T1 or T2; HR 2.13; 95% CI 1.13 to 4.02; p=0.02) and ER-negative status (ER status positive versus negative; HR 0.34; 95% CI 0.18 to 0.65; p=0.001) were associated with statistically significant greater risk of distant relapse or death.

TABLE 5
MULTIVARIATE Cox Regression Analysis
of Association with DRFS

| | Validation Cohort | |
|---|---|---|
| | (N = 183)* | P |
| Factor | Hazard Ratio (95% CI) | value |
| Age (>50 vs ≦50) | 0.53 (0.27 to 1.04) | 0.063 |
| Clinical Nodal Status (pos vs neg) | 1.76 (0.84 to 3.67) | 0.134 |
| Clinical Tumor Stage (T3 or T4 vs T1 | 2.13 (1.13 to 4.02) | 0.020 |
| or T2) | | |
| Histologic Grade (3 vs 1 or 2) | 0.64 (0.32 to 1.29) | 0.208 |
| ER Status (IHC positive vs negative) | 0.34 (0.18 to 0.65) | 0.001 |
| Taxane (docetaxel vs paclitaxel) | 0.92 (0.49 to 1.73) | 0.795 |
| Prediction (Rx Sensitive vs Insensitive) | 0.19 (0.07 to 0.56) | 0.002 |

*Fifteen cases were excluded from the multivariate analysis due to incomplete data. Likelihood ratio test for the addition of Genomic Prediction to the model was 13.8 on one degree of freedom, p = 0.0002.
The Hazard Ratio is a measure of the risk of distant relapse or death; vs, versus; ER, estrogen receptor.

### Example 10

### Comparison with Other Predictive Genomic Signatures

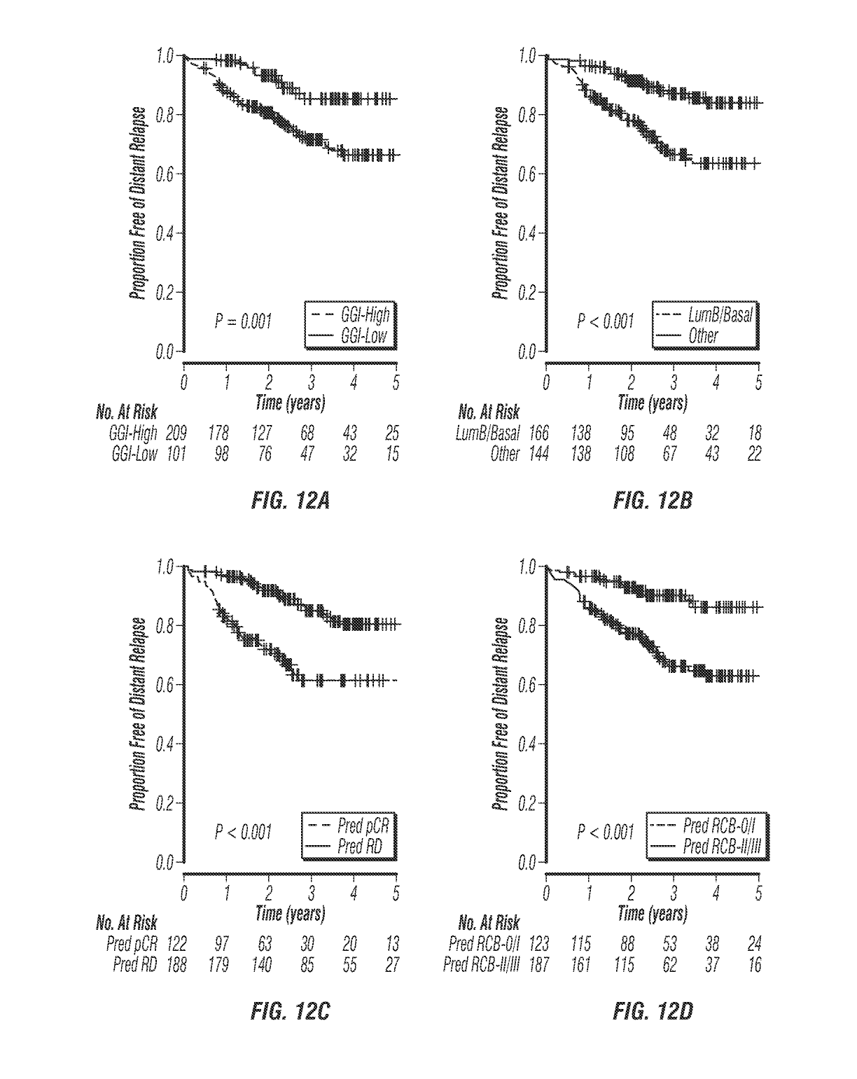

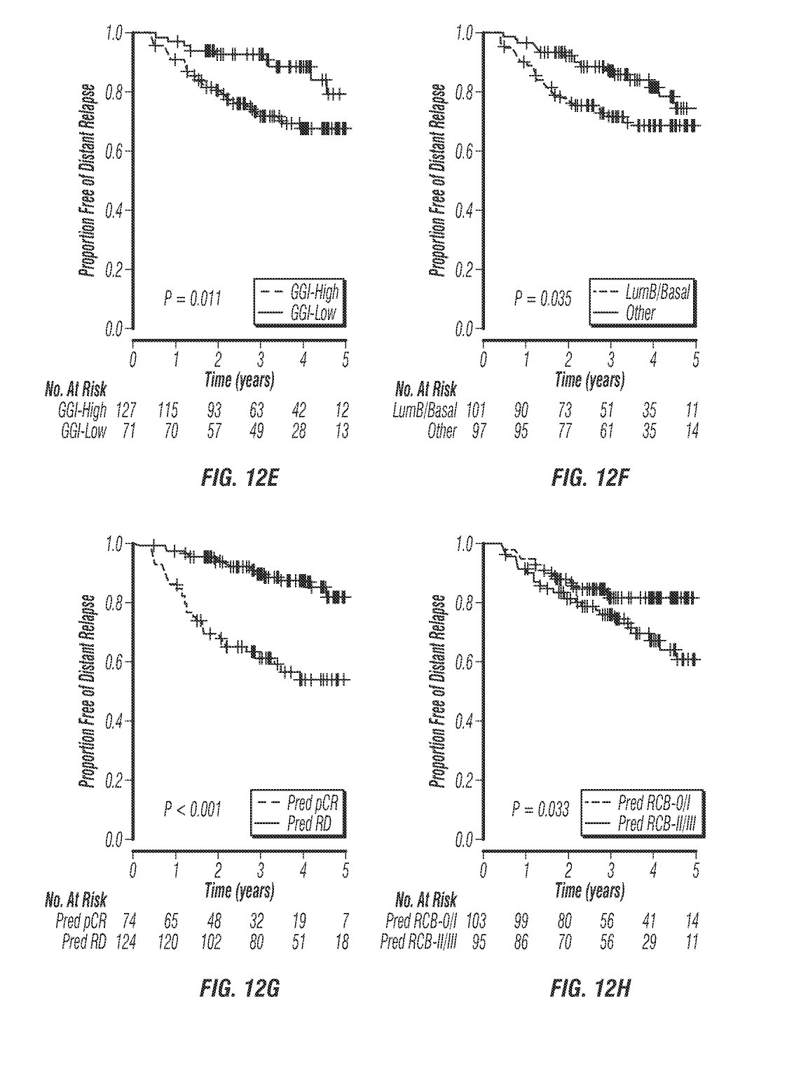

The entire predictive test algorithm described in FIG. 9 had PPV of 56% (95% CI 31 to 78) for pathologic response prediction in the validation cohort (Table 6) after excluding patients with predicted endocrine sensitivity (high or intermediate SET). We also evaluated other phenotypic predictors that have published association with higher probability of pCR to neoadjuvant chemotherapy, have a pre-defined threshold for prediction of pCR that was based on Affymetrix microarray data, and that we have confirmed to be correctly calculated in our hands: the 96-gene genomic grade index (GGI) to define high versus low grade (high GGI predicted pCR) (Liedtke et al., 2009), a 52-gene signature (PAM50) to assign intrinsic subtype (basal-like, HER2 and luminal B subtypes predicted pCR) (Parker et al., 2009), and a 30-gene signature (DLDA30) developed to predict pCR versus residual disease (Hess, Anderson et al., 2006). These tests were significantly predictive of pathologic response in the discovery cohort (lower 95% confidence limit of the PPV greater than the baseline pCR rate of 19% and pCR or RCB-I rate of 29%), and the tests had NPV of 84% or greater (Table 6). Performance in the validation cohort was similar, but not all tests had PPV and NPV that was significantly greater than the baseline response rates (pCR rate of 25% and pCR or RCB-I rate of 30%). The entire prediction algorithm (FIG. 9), demonstrated significantly better DRFS for patients who were predicted to be treatment-sensitive (Table 6). The other tests (GGI, PAM50, DLDA30) demonstrated worse DRFS for patients who were predicted to have chemosensitive breast cancer (FIG. 12), as indicated by their negative ARR (Table 6).

The performance of the different genomic signatures for predicting 3-year DRFS was compared on the basis of the diagnostic likelihood ratio (DLR), which is clinically useful statistic for summarizing the diagnostic accuracy of tests (Deeks and Altman, 2004). The DLR+ summarizes how many times a positive test (predicted distant relapse or treatment insensitive) is more likely among patients who experience distant metastasis within 3 years, compared to those who do not. The DLR− is a similar metric for a negative test (predicted absence of relapse or treatment sensitive), which is more relevant in the context of this test. A clinically useful test associated with the presence of relapse should have DLR+>1, whereas a test associated with the absence of relapse should have DLR−<1. Another useful property of the DLR is that it allows calculation of the post-test odds of relapse, simply by multiplying the pre-test odds of relapse by the DLR. The odds ratio (OD), defined as DLR+/DLR−, is also related to the coefficient of a logistic regression model of the binary genomic test for predicting the binary relapse outcome. The values summarized in Table 7 were calculated from the K-M estimates of DRFS for the two predicted groups from each genomic predictor, for the overall validation cohort and for the ER-positive and ER-negative subsets.

The predictive test of Example 9 (last entry in Table 7) is the only test with a significant DLR− (0.33, 0.27, 0.35 in the overall validation cohort and ER+, ER− subsets), indicating a 3-fold reduction in the odds of distant relapse in the presence of a negative test result (predicted treatment sensitive). The DLR+ of the genomic predictor was >1 in all 3 cohorts, but was not significant. The ER-stratified predictor of pCR/RCB-I showed consistent but not significant metrics. The first three genomic predictors showed paradoxical statistics (DLR+<1 and DLR−>1), i.e. a positive test result (predicted relapse) was associated with lower odds of relapse and vice versa.

TABLE 6
Performance of Genomic Signatures for Predicting Pathologic Response and 3-year
DRFS

| | | | | | | | Prediction of Distant Relapse or Death | | | | | |
|---|---|---|---|---|---|---|---|---|---|---|---|---|
| | | | | | | | Within 3 Years | | | | | |
| | Prediction of Pathologic Response | | | | | | Discovery Cohort | | | Validation Cohort | | |
| | Discovery Cohort | | | Validation Cohort | | | (N = 310) | | | (N = 198) | | |
| | N | | % | N | | % | % NPV | | | % NPV | | |
| Predictor | (% Resp) | % PPV | NPV | (% Resp) | % PPV | NPV | (DRFS) | % PPV | % ARR | (DRFS) | % PPV | % ARR |
| Genomic | 301 | 36 | 88 | 101 | 40 | 84 | 72 | 14 | −14 | 72 | 7 | −21 |
| Grade | (29) | (30 to | (79 to | (30) | (28 to | (70 to | (65 to | (6 to | (−25 to | (64 to | (1 to | (−30 to |
| Index | | 43) | 93) | | 54) | 93) | 79) | 22) | −3) | 80) | 13) | −10) |
| (High) | | | | | | | | | | | | |
| Genomic | 301 | 40 | 85 | 101 | 40 | 78 | 66 | 13 | −20 | 72 | 12 | −16 |
| Subtype | (29) | (32 to | (78 to | (30) | (25 to | (65 to | (58 to | (7 to | (−31 to | (62 to | (6 to | (−27 to |
| Classifier | | 48) | 90) | | 56) | 87) | 76) | 19) | −10) | 81) | 20) | −5) |
| (Luminal B | | | | | | | | | | | | |
| or Basal- | | | | | | | | | | | | |
| like) | | | | | | | | | | | | |
| Genomic | 301 | 46 | 83 | 101 | 40 | 75 | 62 | 15 | −24 | 62 | 10 | −28 |
| Predictor | (29) | (37 to | (77 to | (30) | (24 to | (63 to | (52 to | (9 to | (−36 to | (50 to | (4 to | (−41 to |
| of pCR | | 55) | 88) | | 58) | 85) | 73) | 20) | −12) | 73) | 16) | −−16) |
| ER- | 301 | 69 | 100 | 101 | 42 | 81 | 85 | 30 | 15 | 82 | 24 | 5 |
| stratified | (29) | (60 to | (98 to | (30) | (28 to | (68 to | (78 to | (22 to | (4 to | (74 to | (14 to | (−7 to |
| Genomic | | 77) | 100) | | 57) | 91) | 93) | 37) | 25) | 90) | 32) | 16) |
| Predictor | | | | | | | | | | | | |
| of | | | | | | | | | | | | |
| pCR/RCB-I^{§} | | | | | | | | | | | | |
| Predictive | 256 | 78 | 84 | 91 | 56 | 73 | 95 | 36 | 31 | 92 | 25 | 18 |
| Test (Rx | (31) | (66 to | 78 to | (33) | (31 to | (61 to | (91 to | (27 to | (22 to | (85 to | (18 to | (6 to |
| Sensitive)^{§¶#} | | 88) | 89) | | 78) | 82) | 100) | 44) | 41) | 100) | 33) | 28) |

N, number or patients evaluated;
%, percent;
Resp, pathologic response rate;
PPV, positive predictive value;
NPV, negative predictive value;
DRFS, distant relapse-free survival estimate at 3 years;
ARR, absolute risk reduction for event within 3 years if predicted to be treatment-sensitive (−, any negative risk reduction was in favor of predicted treatment-insensitive). The 95% confidence intervals (parentheses) for PPV and NPV for prediction of pathologic response were based on binomial approximation.
^{§}Performance of the pCR predictor on the discovery cohort is optimistically biased because the predictor was trained on a subset of these samples. Performance of the pCR/RCB-I predictor and of the overall genomic prediction test on the discovery cohort represents resubstitution performance, since the predictors were trained on the same cohort.
^{¶}Genomic prediction of pathologic response was evaluated in the SET-Low subset in both cohorts.
^{#}Performance of the predictive test is optimistically biased in the discovery cohort because a component of the test was trained on DRFS events to define resistance.

TABLE 7
Comparison of Genomic Signatures Performance for Predicting 3-year DRFS

| | | | | ER-positive Subset | | | ER-negative Subset | | |
|---|---|---|---|---|---|---|---|---|---|
| | | | | (N = 123) | | | (N = 74) | | |
| | Validation Cohort (N = 198) | | | DLR+ | DLR− | OR | DLR+ | DLR− | OR |
| | DLR+ | DLR− | OR | (95% | (95% | (95% | (95% | (95% | (95% |
| Predictor | (95% CI) | (95% CI) | (95% CI) | CI) | CI) | CI) | CI) | CI) | CI) |
| Genomic | .30 | 1.50 | .2 | .62 | 1.41 | .44 | .17 | 1.20 | .14 |
| Grade | (.06 to | (.97 to | (.04 to | (.13 to | (.45 to | (.06 to | (.0 to | (.73 to | (.0 to |
| Index | 0.63) | 2.20) | .47) | 1.33) | 2.88) | 1.71) | .77) | 2.01) | .70) |
| (High) | | | | | | | | | |
| Genomic | .55 | 1.53 | .36 | .81 | 1.32 | .62 | .60 | 1.21 | .50 |
| Subtype | (.24 to | (.97 to | (.14 to | (.23 to | (.36 to | (.15 to | (.15 to | (.69 to | (.12 to |
| Classifier | .96 | 2.33) | .74) | 1.60) | 2.64) | 2.80) | 1.56) | 2.14) | 1.50) |
| (Luminal B | | | | | | | | | |
| or Basal- | | | | | | | | | |
| like) | | | | | | | | | |
| Genomic | .43 | 2.39 | .18 | .86 | 1.98 | .43 | .28 | 1.31 | .21 |
| Predictor of | (.18 to | (1.41 to | (.07 to | (.33 to | (.01 to | (.10 to | (.0 to | (.77 to | (.0 to |
| pCR | .73) | 3.96) | .37) | 1.50) | 6.37) | 88.1) | .92) | 2.25) | .77) |
| ER- | 1.18 | .85 | 1.39 | 1.07 | .93 | 1.15 | 1.70 | .68 | 2.50 |
| stratified | (.65 to | (.46 to | (.66 to | (.35 to | (.16 to | (.31 to | (.79 to | (.32 to | (.88 to |
| Genomic | 1.83) | 1.36) | 2.93) | 2.11) | 2.03) | 6.61) | 3.65) | 1.27) | 7.03) |
| Predictor of | | | | | | | | | |
| pCR/RCB-I | | | | | | | | | |
| Predictive | 1.32 | .33 | 4.01 | 1.33 | .27 | 4.88 | 1.33 | .35 | 3.78 |
| Test (Rx | (.84 to | (.07 to | (1.55 to | (.56 to | (.01 to | (1.05 to | (.76 to | (.01 to | (1.16 to |
| Sensitive) | 1.93) | .78) | 21.6) | 2.34) | 0.98) | 206) | 2.30) | .99) | 138) |

DLR: Diagnostic likelihood ratio;
DLR+: DLR given a positive test result (predicted treatment insensitive);
DLR−: DLR given a negative test result (predicted treatment sensitive);
OR: odd ratio of a positive test result over a negative test result (DLR+/DLR−);
CI: confidence interval. Confidence intervals were calculated through bootstrap with 999 iterations

### Example 11

### Analysis of Patient Samples Using Predictor for Assessing Outcome of Therapy

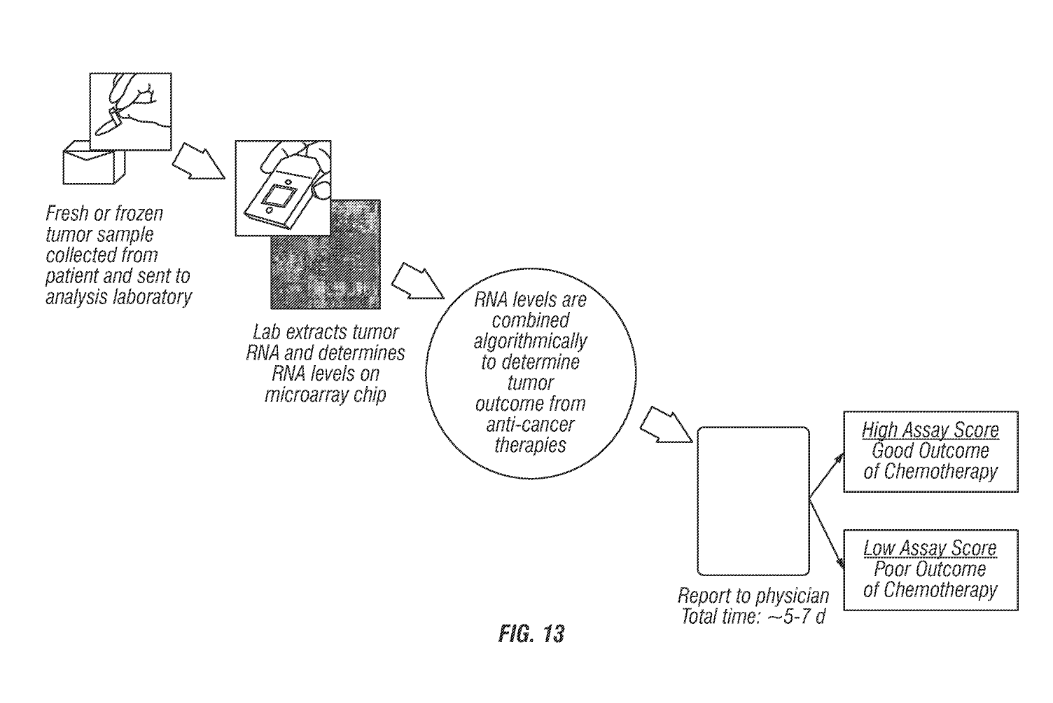

FIG. 13 shows a schematic of how a patient sample may be collected at the time of biopsy or at the time of surgery, and analyzed in a laboratory to produce a result from the predictor to be used to assess likely outcome of chemotherapy. A tumor sample, collected as a needle biopsy or a fresh tumor sample from the excised tumor after surgery is added to a pre-supplied tube containing RNA preservative solution. The tube is shipped overnight to a qualified laboratory for analysis of gene expression.

RNA is extracted in a manner described in Example 1. A gene chip such as Affymetrix U133A (Affymetrix, Inc., Santa Clara, Calif.) is used to analyze the expression levels of genes of Tables 2, 3 and 4. The resulting expression values are then normalized as described in Examples 2, 4, and 8, and weighted according to their respective coefficients to calculate the predictor score. Using cut-off values for the predictor score, a patient's tumor can be classified as either a High Score (good outcome from therapy) or a Low Score (poor outcome of therapy). The analyses could be completed within 5-7 days from receipt of a tumor sample to provide a report on results to the requesting physician. Decisions may be made by physicians regarding the inclusion of a certain therapy if the likely outcome is good or alternatively, to consider additional aggressive therapy regimens for the patient in the likely event of a poor outcome.

### REFERENCES

The following references, to the extent that they provide exemplary procedural or other details supplementary to those set forth herein, are specifically incorporated herein by reference.
Armitage, P., G. Berry & J. N. S. Matthews (2002). Statistical Methods In Medical Research, Fourth Edition. Blackwell Science.
Ayers, M., W. F. Symmans, et al. (2004). “Gene expression profiles predict complete pathologic response to neoadjuvant paclitaxel and fluorouracil, doxorubicin, and cyclophosphamide chemotherapy in breast cancer.” J Clin Oncol 22(12): 2284-93.
Bear, H. D., S. Anderson, et al. (2006). “Sequential preoperative or postoperative docetaxel added to preoperative doxorubicin plus cyclophosphamide for operable breast cancer: National Surgical Adjuvant Breast and Bowel Project Protocol B-27.” J Clin Oncol 24(13): 2019-27.
Bild, A. H., G. Yao, et al. (2006). “Oncogenic pathway signatures in human cancers as a guide to targeted therapies.” Nature 439(7074): 353-7.
Carey, L. A., R. Metzger, et al. (2005). “American Joint Committee on Cancer tumor-node-metastasis stage after neoadjuvant chemotherapy and breast cancer outcome.” J Natl Cancer Inst 97(15): 1137-42.
Carlson, R. W., B. O. Anderson, et al. (2000). “NCCN practice guidelines for breast cancer.” Oncology (Williston Park) 14(11A): 33-49.
Chang, J. C., E. C. Wooten, et al. (2003). Gene expression profiling for the prediction of therapeutic response to docetaxel in patients with breast cancer. Lancet 362(9381): 362-9.
Deeks J J, Altman D G. Diagnostic tests 4: likelihood ratios. BMJ. Jul. 17, 2004; 329(7458):168-169
Dudoit, S., J. Fridlyand, et al. (2002). “Comparison of discrimination methods for the classification of tumors using gene expression data.” J Am Stat Assoc 97: 77-87.
Fisher, B., J. Bryant, et al. (1998). “Effect of preoperative chemotherapy on the outcome of women with operable breast cancer.” J Clin Oncol 16(8): 2672-85.
Goldhirsch, A., W. C. Wood, et al. (2003). “Meeting highlights: updated international expert consensus on the primary therapy of early breast cancer.” J Clin Oncol 21(17): 3357-65.
Hennessy, B. T., G. N. Hortobagyi, et al. (2005). “Outcome after pathologic complete eradication of cytologically proven breast cancer axillary node metastases following primary chemotherapy.” J Clin Oncol 23(36): 9304-11.
Hennessy, B. T. and L. Pusztai (2005). “Adjuvant therapy for breast cancer.” Minerva Ginecol 57(3): 305-26.
Hess, K. R., K. Anderson, et al. (2006). “Pharmacogenomic predictor of sensitivity to preoperative chemotherapy with paclitaxel and fluorouracil, doxorubicin, and cyclophosphamide in breast cancer.” J Clin Oncol 24(26): 4236-44.
Jackson J E, Mudholkar, G S. (1979). “Control procedures for residuals associated with principal component analysis.” Tehcnometrics 21:341-349.
Kaufmann, M., G. N. Hortobagyi, et al. (2006). “Recommendations from an international expert panel on the use of neoadjuvant (primary) systemic treatment of operable breast cancer: an update.” J Clin Oncol 24(12): 1940-9.
Kuerer, H. M., L. A. Newman, et al. (1999). “Clinical course of breast cancer patients with complete pathologic primary tumor and axillary lymph node response to doxorubicin-based neoadjuvant chemotherapy.” J Clin Oncol 17(2): 460-9.
Kuroi, K., M. Toi, et al. (2005). “Unargued issues on the pathological assessment of response in primary systemic therapy for breast cancer.” Biomed Pharmacother 59 Suppl 2: S387-92.
Kurosumi, M. (2004). “Significance of histopathological evaluation in primary therapy for breast cancer—recent trends in primary modality with pathological complete response (pCR) as endpoint.” Breast Cancer 11(2): 139-47.
Lai, C., M. J. Reinders, et al. (2006). “A comparison of univariate and multivariate gene selection techniques for classification of cancer datasets.” BMC Bioinformatics 7(1): 235.
Liedtke C, Hatzis C, Symmans W F, et al. Genomic grade index is associated with response to chemotherapy in patients with breast cancer. J Clin Oncol. Jul. 1, 2009; 27(19):3185-3191.
Ma, S., X. Song, et al. (2006). “Regularized binormal ROC method in disease classification using microarray data.” BMC Bioinformatics 7: 253.
Parker J S, Mullins M, Cheang M C, et al. Supervised risk predictor of breast cancer based on intrinsic subtypes. J Clin Oncol. Mar. 10, 2009; 27(8):1160-1167.
Perou, C. M., T. Sorlie, et al. (2000). “Molecular portraits of human breast tumours.” Nature 406(6797): 747-52.
Pusztai, L., M. Ayers, et al. (2003). “Gene expression profiles obtained from fine-needle aspirations of breast cancer reliably identify routine prognostic markers and reveal large-scale molecular differences between estrogen-negative and estrogen-positive tumors.” Clin Cancer Res 9(7): 2406-15.
Pusztai, L., M. Ayers, et al. (2003). “Clinical application of cDNA microarrays in oncology.” Oncologist 8(3): 252-8.
Pusztai, L., C. Sotiriou, et al. (2003). “Molecular profiles of invasive mucinous and ductal carcinomas of the breast: a molecular case study.” Cancer Genet Cytogenet 141(2): 148-53.
Rajan, R., A. Poniecka, et al. (2004). “Change in tumor cellularity of breast carcinoma after neoadjuvant chemotherapy as a variable in the pathologic assessment of response.” Cancer 100(7): 1365-73.
Ross, J. S., J. A. Fletcher, et al. (2003). “HER-2/neu testing in breast cancer.” Am J Clin Pathol 120 Suppl: S53-71.
Ross, J. S., J. A. Fletcher, et al. (2003). “The Her-2/neu gene and protein in breast cancer 2003: biomarker and target of therapy.” Oncologist 8(4): 307-25.
Ross, J. S., G. P. Linette, et al. (2003). “Breast cancer biomarkers and molecular medicine.” Expert Rev Mol Diagn 3(5): 573-85.
Rouzier, R., C. M. Perou, et al. (2005). “Breast cancer molecular subtypes respond differently to preoperative chemotherapy.” Clin Cancer Res 11(16): 5678-85.
Rouzier, R., L. Pusztai, et al. (2005). “Nomograms to predict pathologic complete response and metastasis-free survival after preoperative chemotherapy for breast cancer.” J Clin Oncol 23(33): 8331-9.
Rouzier, R., R. Rajan, et al. (2005). “Microtubule-associated protein tau: a marker of paclitaxel sensitivity in breast cancer.” Proc Natl Acad Sci USA 102(23): 8315-20.
Rouzier, R., P. Wagner, et al. (2005). “Gene expression profiling of primary breast cancer.” Curr Oncol Rep 7(1): 38-44.
Stec, J., J. Wang, et al. (2005). “Comparison of the predictive accuracy of DNA array-based multigene classifiers across cDNA arrays and Affymetrix GeneChips.” J Mol Diagn 7(3): 357-67.
Symmans, W. F., M. Ayers, et al. (2003). “Total RNA yield and microarray gene expression profiles from fine-needle aspiration biopsy and core-needle biopsy samples of breast carcinoma.” Cancer 97(12): 2960-71.
Symmans, W. F., F. Peintinger, et al. (2007). “Measurement of Residual Breast Cancer Burden to Predict Survival After Neoadjuvant Chemotherapy.” J Clin Oncol.
Tibshirani R. J. (2009) Univaraite shrinkage in the Cox model for high dimensional data. Statistical Applications in Genetics and Molecular Biology 8(1): article 21.
van 't Veer, L. J., H. Dai, et al. (2002). “Gene expression profiling predicts clinical outcome of breast cancer.” Nature 415(6871): 530-6.
van de Vijver, M. J., Y. D. He, et al. (2002). “A gene-expression signature as a predictor of survival in breast cancer.” N Engl J Med 347(25): 1999-2009.
Wang, Y., J. G. Klijn, et al. (2005). “Gene-expression profiles to predict distant metastasis of lymph-node-negative primary breast cancer.” Lancet 365(9460): 671-9.

1. A method of evaluating a cancer patient comprising the steps of:
(a) evaluating gene expression levels in a patient sample comprising cancer cells or an RNA sample isolated from such a patient sample, wherein a plurality of genes to be evaluated are selected from one or more of Table 2, Table 3, and Table 4;
(b) calculating a predictor score using the gene expression levels; and
(c) assessing the likelihood of a therapeutic outcome using the predictor score.

2. The method of claim 1, further comprising identifying a cancer patient with a disease state classified as a residual disease state prior to evaluation.

3. The method of claim 1, wherein the therapeutic outcome distant relapse-free survival (DRFS).

4. The method of claim 1, wherein the transcriptional profile index comprises 5 or more genes of Table 2, Table 3, and Table 4.

5-7. (canceled)

8. The method of claim 1, wherein the transcriptional profile index comprises 60 genes of Table 2, Table 3, and Table 4.

9. The method of claim 1, wherein the transcriptional profile index comprises all genes of Table 2, Table 3, and Table 4.

10. The method of claim 1, further comprising determining Her2-neu and/or estrogen receptor status of the patient sample.

11. The method of claim 1, wherein the predictor score includes evaluation of tumor size, cellularity of tumor bed, and/or nodal burden.

12. The method of claim 1, further comprising providing a treatment recommendation depending on the predictor classification.

13. The method of claim 12, wherein the treatment is a combination of one or more cancer therapy.

14. The method of claim 13, wherein the treatment is hormonal therapy and/or chemotherapy.

15. The method of claim 14, wherein the chemotherapy consists of taxane and anthracycline therapy.

16. The method of claim 1, wherein preparing the predictor score comprises the steps of:
(a) obtaining data associated with a plurality of breast cancer patients comprising measuring expression levels of a plurality of genes in samples from the patients;
(b) partitioning the data into a first and second dataset;
(c) evaluating the data and identifying data associated with a particular treatment outcome;
(d) selecting a set of genes whose expression levels are indicative of therapeutic outcome

17. The method of claim 16, wherein the index includes evaluation of survival of the patient population sampled for all or part of the reference population of tumor samples.

18. The method of claim 17, wherein the method includes evaluation of distant relapse-free survival (DRFS) of the patient population.

19. A kit to determine responsiveness of a cancer comprising:
(a) reagents for determining expression levels of a plurality of genes selected from Table 2, Table 3, and Table 4 or combinations thereof; and
(b) software encoding an algorithm for calculating a predictor score based on the analysis of the gene expression levels.

20. A system for providing assessment of a sample relative to a gene expression index, the system comprising:
(a) an application server comprising an input manager to receive expression data from a user for a plurality of genes selected from Table 2, Table 3, and Table 4 or combinations thereof obtained from a patient sample or an RNA sample from such patient sample; and
(b) a network server comprising an output manager constructed and arranged to provide an assessment to the user.

21. A computer readable medium having software modules for performing the method of claim 1 comprising the acts of:
(a) comparing gene expression data obtained from a patient sample for a plurality of genes selected from Table 2, Table 3, and Table 4 or combinations thereof with a reference; and
(b) providing a predictor score to a physician for use in determining an appropriate therapeutic regimen for a patient.

22. A computer system, having a processor, memory, external data storage, input/output mechanisms, a display, for performing the method of claim 1, comprising:
(a) a database;
(b) logic mechanisms in the computer for generating the transcriptional profile index; and
(c) a comparing mechanism in the computer for comparing the gene expression reference to expression data from a patient sample or an RNA sample from such a patient sample to calculate a predictor score.

23. An internet accessible portal for providing biological information constructed and arranged to execute a computer-implemented method of claim 1 for providing:
(a) a comparison of gene expression data of a plurality of genes of claim 1 in a patient sample with a transcriptional profile index; and
(b) providing a predictor score to a physician for use in determining an appropriate therapeutic regime for a patient.
